(19) United States
(12) Patent Application Publication
(10) Pub. No.: US 2026/0061155 A1
(43) Pub. Date: Mar. 5, 2026

(54) VOICE CONTROLLED EYE MASSAGER

(71) Applicant: Reestar International Limited, HONG KONG (HK)

(72) Inventor: Wesley King Juen Tong, Hong Kong (HK)

(21) Appl. No.: 18/986,545

(22) Filed: Dec. 18, 2024

Related U.S. Application Data
(63) Continuation-in-part of application No. 18/824,459, filed on Sep. 4, 2024.

(51) Int. Cl. A61M 21/02; A61H 23/02; A61M 21/00

(52) U.S. Cl. CPC A61M 21/02; A61H 23/02; A61H 2201/0107; A61H 2201/0207; A61H 2201/1607; A61H 2201/165; A61H 2201/5007; A61H 2201/5043; A61H 2201/5048; A61H 2205/024; A61M 2021/0022; A61M 2205/3303

(57) ABSTRACT

Disclosed herein are computing system, apparatus, article of manufacture, method and/or computer program product embodiments, and/or combinations and sub-combinations and sub-combinations thereof, for providing a state of a user, via a display device of an eye massager, while the eye massager performs one or more massage operations on one or more portions of a head or face of a user. For example, a computer-implemented method may include obtaining sensor data from one or more sensors of the eye massager; while the one or more massage components perform the one or more massage operations to the one or more portions of the head of the user, determining a state of the user based on the sensor data, the state of the user indicating a stress level of the user; and causing a display device of the eye massager to output information associated with the state of the user.

### CROSS REFERENCE TO RELATED APPLICATIONS

This application is a Continuation-in-Part (CIP) of U.S. application Ser. No. 18/824,459, filed Sep. 4, 2024, which is hereby incorporated by reference in its entirety.

### BACKGROUND

### Field

This disclosure is generally directed to massagers, and more particularly to voice controlled eye massagers.

### Summary

Provided herein are system, apparatus, article of manufacture, method and/or computer program product embodiments, and/or combinations and sub-combinations thereof, for providing a state of a user, via a display device of an eye massager, while the eye massager performs one or more massage operations on one or more portions of a head or face of a user. In some examples, an eye massager may include a display device, a first housing element, a second housing element, one or more massage components, a memory storing instructions, and at least one processor coupled to the memory. The display device may be coupled to the second housing element and the first housing element may be coupled to the second housing element with the display device being between the first housing element and the second housing element. The at least one processor may be configured to execute the instructions to determine one or more massage operations for a user; cause the one or more massage components to perform one or more massage operations to one or more portions of a head of the user; determine a state of the user, the state of the user indicating a stress level of the user while the one or more massage components perform the one or more massage operations to the one or more portions of the head of the user; and cause the display device to output information associated with the state of the user.

In some aspects, an eye massager is provided for providing a state of a user, via a display device of an eye massager, while the eye massager performs one or more massage operations on one or more portions of a head or face of a user. The eye massager may include a display device a body, one or more massage components, a memory storing instructions, and at least one processor coupled to the memory. The display device may be coupled to the body. The at least one processor may be configured to execute the instructions to determine one or more massage operations for a user; cause the one or more massage components to perform one or more massage operations to one or more portions of a head of the user; determine a state of the user, the state of the user indicating a stress level of the user while the one or more massage components perform the one or more massage operations to the one or more portions of the head of the user; and cause the display device to output information associated with the state of the user.

In other aspects, a computer-implemented method is provided for providing a state of a user, via a display device of an eye massager, while the eye massager performs one or more massage operations on one or more portions of a head or face of a user. The computer-implemented method may include determining one or more massage operations; causing one or more massage components of an eye massager to perform the one or more massage operations to one or more portions of a head of a user; obtaining sensor data from one or more sensors of the eye massager; determining a state of the user based on the sensor data, the state of the user indicating a stress level of the user while the one or more massage components perform the one or more massage operations to the one or more portions of the head of the user; and causing a display device of the eye massager to output information associated with the state of the user.

### BRIEF DESCRIPTION OF THE FIGURES

The accompanying drawings are incorporated herein and form a part of the specification.

FIG. 1 illustrates a block diagram of an example electronic eye massager, according to some examples of the present disclosure;

FIG. 2 illustrates an example diagram of a user wearing the example electronic eye massager, according to some examples of the present disclosure;

FIG. 3A illustrates an exploded view of the example electronic eye massager, according to some examples of the present disclosure;

FIGS. 3B-3D illustrate one or more example airbags of the example electronic eye massager, according to some examples of the present disclosure;

FIG. 3E illustrates an example heating device of the example electronic eye massager, according to some examples of the present disclosure;

FIG. 3F illustrates an exploded view of an example electronic eye massager with one or more components of a display system, according to some examples of the present disclosure;

FIGS. 3G-3P illustrate diagrams of an example electronic eye massager with one or more modular components;

FIG. 3Q illustrates an isometric rear view of an example electronic eye massager with one or more components of display system, according to some examples of the present disclosure;

FIG. 3R illustrates a rear view of an example electronic eye massager, according to some examples of the present disclosure;

FIG. 4 is a flow chart of an example process for controlling the example electronic eye massager with spoken commands, according to some examples of the present disclosure;

FIG. 5 is a flow chart of an example process for providing information of a user while one or more massage operations or a mode of massage is being performed by an example electronic eye massager, according to some examples of the present disclosure;

FIG. 6 is a diagram illustrating an example of a neural network architecture, according to some examples of the present disclosure; and

FIG. 7 illustrates an example computer system that can be used for implementing various aspects of the present disclosure.

In the drawings, like reference numbers generally indicate identical or similar elements. Additionally, generally, the left-most digit(s) of a reference number identifies the drawing in which the reference number first appears.

### DETAILED DESCRIPTION

An electronic eye massager (“eye massager” hereinafter) may include one or more motors or air pumps and one or more compartments or airbags. In some examples, the airbags of the eye massager may be inflated or deflated by the air pumps. The airbags may apply pressure and perform a massage to one or more portions of a head of a user wearing the eye massager, such as around the left eye, right eye, left temple, and/or right temple of the user, when inflating or deflating. As described herein, the applied pressure and performed massage may relieve eye strain and fatigue, improve blood circulation to the eyes, reduce headaches caused by eye strain, promote better sleep, alleviate sinus pressure and/or reduce intraocular pressure. In some cases, the eye massager may be configured to perform one or more massage operations. In some instances, the eye massager may be configured to perform one or more modes of massages and each mode of massage may include one or more massage operations. Moreover, the massage operations may be associated with an inflation and/or deflation pattern, one or more levels of pressure (e.g., pounds per square inch or “psi”) and duration of massage operations.

In some cases, the eye massagers may include a user interface, such as one or more buttons, that enable a user to initiate the massage operations, or adjust one or more parameters or settings of the massage operations or mode of massage currently being performed by the eye massagers (e.g., level of pressure (e.g., pounds per square inch or “psi”) and/or duration). However, when the user wears or uses the eye massager, the eye massager covers the eyes of the user and obstructs the vision of the user. In such examples, the user may not be able to see the user interface of the eye massager and may have trouble using the user interface to initiate a massage mode, a massage operation or adjust the parameters or settings of the massage mode or massage operation. The experience of the user may be diminished due to the user needing to remove the eye massager in order to interact or engage with the user interface to initiate the mode of massage, the massage operation or adjust the parameters or settings of the mode of massage or massage operation.

Provided herein are a system, apparatus, device, method, and/or computer program product embodiment, and/or combinations and sub-combinations thereof (“systems and technique” hereinafter), for obtaining, from a user, audio signals associated with the user speaking and initiating one or more massage operations, a mode of massage or adjusting one or more parameters or settings of the massage operations or mode of massage currently being performed by an eye massager. The user may be able to initiate the mode of massage, the massage operation, or adjust the parameters or settings of the massage operations currently being performed by the eye massager without removing the eye massager to engage with the user interface of the eye massager. In some examples, the eye massager may include a voice recognition module. The voice recognition module may enable the eye massager to receive an audio signal of one or more words or phrases spoken by a user. Moreover, the voice recognition module may determine one or more massage operations and/or one or more adjustments to the massage operations currently being performed by the eye massager based on the audio signal. Further, the eye massager may initiate the determined massage operations and/or adjust the parameters or settings of the massage operations currently performed by the eye massager in accordance with the determined adjustments.

In some aspects, the one or more compartments or airbags of the eye massager may only engage with portions of the face around the left and right eye of a user or only partially cover the right and/or left temple of the user, when the user uses or wears the eye massager. In such aspects, the health benefits and/or experience of the user may be diminished as the compartments or airbags may not fully engage with the entire left and right temple of the user. As described herein, the health benefits associated with applied pressure and performed massages to the entire left and right temple may include, muscle tension relief, migraine relief, stress relief and brain stimulation. The eye massager described herein may include a larger or extended compartment or airbag or a greater number of compartments or airbags to cover the left and right temple of a user. That way, the entire left and right temple of the user may be fully engaged with the compartments or airbags of the eye massager.

In some cases, the one or more motors or air pumps of the eye massager that inflate or deflate the one or more compartments or airbags of the eye massager may be loud. In such cases, the experience of the user may be diminished as the massage being received by the eye massager may become less soothing or relaxing due to the loud sounds generated by the motors or air pumps inflating or deflating the compartments or airbags. The eye massager described herein may include a sound reduction device for each of the motors or air pumps of the eye massager. For example, the sound reduction device may include a housing or covering. The housing or covering may cover fully or partially a body of each motor or air pump, such as a silicon housing. In some instances, the housing or covering may be 1 to 2.5 mm in thickness.

In some aspects, the eye massager may be configured to perform one or more heating operations. In some instances, the modes of massages may include one or more heating operations. Moreover, the heating operations may be associated with one or more levels of temperature and duration of the heating operations. As described herein, the heating operations (e.g., the outputted heat or elevated temperatures) may help promote blood circulation of a face of a user.

Additionally, or alternatively, the eye massager may be configured to perform one or more vibrational operations. In some instances, the modes of massages may include one or more vibrational operations. Moreover, the vibrational operations may be associated with a vibrational pattern, one or more levels of frequency and duration of vibrational operations. In some cases, the heating operations and/or vibrational operations may be implemented while and during the implementation of the massage operations.

In some cases, the massage operation(s) or the mode of massage may not be adapted to a state of a user. Examples of the state of the user may include a stressed state, a relaxed state or a stress level. In some instances, the systems and techniques provided herein may process sensor data associated with a user using the eye massager to determine the massage operation(s) or the mode of massage for the user. For example, the systems and techniques provided herein may process the sensor data to determine biometric information of the user. Based on the biometric information, the systems and techniques provided herein may determine a state of the user. Based on the state of the user, the systems and techniques provided herein may determine one or more parameters or settings for the massage operation(s) or the mode of massage. In some aspects, the eye massager may initiate the determined massage operation(s) or mode of massage and/or adjust the parameters or settings of the massage operations currently performed by the eye massager in accordance with the determined parameter(s) or setting(s).

Additionally, or alternatively, the systems and techniques provided herein may process sensor data associated with a user using the eye massager to determine one or more adjustments to the massage operation(s) or mode of massage currently being performed by the eye massager on the user. For example, the systems and techniques provided herein may process sensor data to determine biometric information of the user. Based on the biometric information, the systems and techniques provided herein may determine a state of the user. Based on the state of the user, the systems and techniques provided herein may determine adjustment(s) to the parameter(s) or setting(s) of the massage operation(s) or the mode of massage the eye massager may be performing. In some instances, the eye massager may initiate the determined adjustments(s) in accordance with the determined adjustments to the parameter(s) or setting(s).

In some aspects, the eye massager may include a sensor system. The sensor system may include one or more sensing components or sensors that interface with one or more portions of a head of a user. The sensing components or sensors may generate the sensor data the systems and techniques provided herein may use to determine the biometric information and/or the state of the user.

In some examples, the eye massager may be in a shared or monitored environment. For example, the eye massager may be shared between two individuals or a third-party individual may be monitoring the user while the user is operating or using the eye massager. In such examples, the eye massager may not be able to provide an intuitive or visible means for other users or observers (e.g., caregivers, family members, etc.) to understand a state of the user while the user is using or operating the eye massager.

The systems and techniques provided herein may provide state information associated with the eye massager to the other users or observers. The state information may indicate the state of the user while the user is using or operating the eye massager (e.g., whether the user is relaxed, asleep or stressed during a particular set of massage operations or a mode of massage). In some aspects, the state information may be based on biometric information, as described herein. In some instances, the systems and techniques provided herein may provide the state information of the user to a display system coupled to the exterior surface of the eye massager. The display system may present or display the state information to the other users or observers.

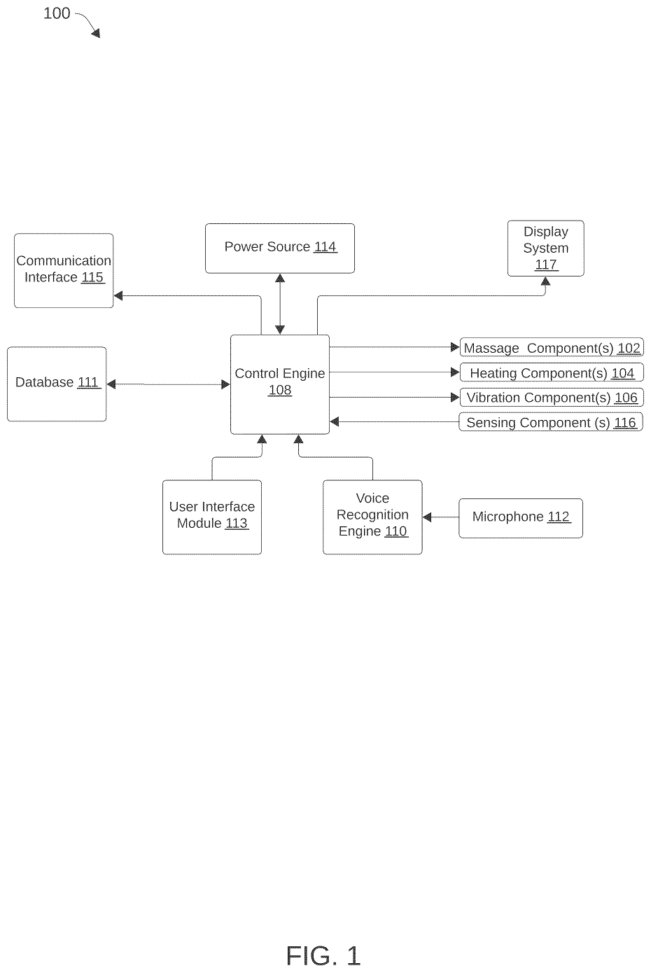

Various embodiments and aspects of this disclosure may be implemented using and/or may be part of eye massager system 100 shown in FIG. 1. It is noted, however, that eye massager system 100 is provided solely for illustrative purposes and is not limiting. Examples and embodiments of this disclosure may be implemented using, and/or may be part of, environments different from and/or in addition to the eye massager system 100, as will be appreciated by persons skilled in the relevant art(s) based on the teachings contained herein. An example of the eye massager system 100 shall now be described.

### Eye Massager

Referring to FIG. 1, example eye massager system 100 may be configured to perform any of the example processes described herein. In some examples, and without limitation, eye massager system 100 may obtain audio signals of one or more words or phrases spoken by a user. Moreover, eye massager system 100 may determine one or more operations, such as massage operations, heating operations and/or vibrational operations, or a mode of massage based on the audio signals. Further, eye massager system 100 may initiate or perform the determined operations or mode of massage. In some instances, the mode of massage may include a predetermined set of operations, such as one or more massage operations, heating operations and/or vibrational operations. Additionally, or alternatively, eye massager system 100 may determine one or more adjustments to one or more parameters or settings of the operations or the mode of massage being performed by eye massager system 100 based on the audio signals. In such aspects, eye massager system 100 may adjust the one or more parameters or settings of the operations being performed by eye massager system 100 in accordance with the determined adjustments. As described herein, the user may be able to initiate the operations, mode of massage or adjust one or more parameters of the mode of massage currently being performed by the eye massager without needing to remove the eye massager and engaging with a user interface of the eye massager.

In some aspects, each mode of massage or operations eye massager system 100 may be configured to perform may include one or more or a set of massage operations. Moreover, the massage operations may be associated with an inflation and/or deflation pattern, one or more levels of pressure (e.g., pounds per square inch or “psi”) and duration of massage operations. Additionally, or alternatively, each mode of massage or operations eye massager system 100 may be configured to perform may include one or more heating operations and/or one or more vibrational operations. As described herein, the heating operations may be associated with one or more levels of temperature and duration of the heating operations. Moreover, the vibrational operations may be associated with a vibrational pattern, one or more levels of frequency and duration of vibrational operations.

In some examples, eye massager system 100 may include, be part of, and/or be implemented by one or more hardware and/or software systems, such as, for example and without limitation, one or more server computers, datacenters and/or datacenter devices, cloud computing infrastructure devices/components, software containers, virtual machines, computer devices, cloud application services, microcontroller units and/or any other computing systems. As illustrated in FIG. 1, eye massager system 100 may include massage component(s) 102 (e.g., one or more electronic/electric air pumps, nozzles, valves, airbags), heat component(s) 104 (e.g., heating device including a high heat conductivity and/or melting point, such as a copper, brass, silver, aluminum, gold nickel, tungsten, and carbon fiber), vibration component(s) 106 (e.g., an electric/electronic motor with an off-centered weight attached to a rotational shaft of the motor), control engine 108, voice recognition engine 110, database 111, microphone 112, user interface module 113, power source 114, communication interface 115, sensing component(s) 116 and display system 117. In some aspects, control engine 108 and voice recognition engine 109 may each include or represent one or more software models and/or algorithms. For example, control engine 108 and/or voice recognition engine 110 may each include or represent one or more artificial intelligence (AI) or machine learning (ML) processes, algorithms or models, such as but not limited to, natural learning process (NLP) model, automated speech recognition, Hidden Markov models (HMMs), Gaussian Mixture models (GMMs), Deep Neural Networks (DNNs), Recurrent Neural Networks (Rnns), Connectionist Temporal Classification (CTC), Convolutional Neural Networks (CNNs), Transformer-based Models), Transfer Learning, Beam Search Decoding, and/or any other AI/ML model.

In some cases, one or more processors of eye massager system 100 may execute control engine 108 and voice recognition engine 110 to implement or perform one or more operations, a mode of massage, or one or more adjustments to one or more parameters or settings of a mode of massage, massage operation, heating operation and/or vibration operation based on the audio signals detected by microphone 112. As described herein the audio signals may be associated with one or more words, terms or phrases spoken by a user operating eye massager system 100. As illustrated in FIG. 1, voice recognition engine 110 may be electrically or communicatively coupled to microphone 112. Microphone 112 may detect and obtain audio signals from a user and generate audio data based on the obtained audio signals. Further, microphone 112 may transmit or provide the audio data to voice recognition engine 110. Voice recognition engine 110 may determine one or more words, phrases or terms, spoken by the user based on the audio data. In some instances, voice recognition engine 110 may generate term data including the determined words, phrases or terms. Moreover, the voice recognition engine may transmit or provide the term data to control engine 108.

In some aspects, voice recognition engine 110 may apply one or more AI/ML models, such as a speech recognition-based AI/ML model, to the audio data. Based on the application of the one or more AI/ML models to the audio data, voice recognition engine 110 may perform any of the described examples processes to determine one or more words, phrase or terms spoken by the user. In such aspects, voice recognition engine 110 may generate term data including the determined one or more words, phrases or terms spoken by the user.

Moreover, control engine 108 may receive the term data from voice recognition engine 110. In some examples, control engine 108 may determine one or more operations or a mode of massage eye massager system 100 may implement or perform based on the term data. Additionally, or alternatively, control engine 108 may determine adjustments to a parameter or setting of a mode of massage, massage operation, heating operation and/or vibration operation based on the term data. In such examples, control engine 108 may determine whether the one or more words, phrases or terms included in the term data match a known or predetermined set of words, phrases and/or terms. Further, control engine 108 may access database 111 to determine whether the one or more words, phrases and/or terms included in the term data match a known or predetermined set of words, phrases or terms. Database 111 may store command data identifying one or more predetermined words, phrases and/or terms.

In some cases, the one or more predetermined words, phrases and/or terms may be associated with an operation or mode of massage eye massager system 100 may perform. In such cases, the command data may identify, for each of such predetermined words, phrases and/or terms, one or more corresponding types of operations (e.g., massage operation, vibration operation and/or heating operation), or mode of massage eye massager system 100 may perform, and one or more parameters or settings of each corresponding types of the operations. Additionally, or alternatively, the one or more predetermined words, phrases and/or terms may be associated with one or more adjustments to an operation or mode of massage eye massager system 100 is currently performing. In such aspects, the command data may identify, for each of such predetermined words, phrases and/or terms, one or more corresponding adjustments to one or more operations (e.g., massage operation, vibration operation and/or heating operation) eye massager system 100 may be performing, and one or more corresponding parameters or settings eye massager system 100.

In some instances, for each of the words, phrases or terms included in the command data, the command data may identify the corresponding components (e.g., components of massage component(s) 102, heating component(s) 104 and/or vibration component(s) 106) for each of the associated parameters or settings. As described herein, examples of parameters or settings that may be identified in the command data may include, but are not limited to, an inflation and/or deflation pattern (e.g., indicating a duration of inflation, duration of deflation, a duration the corresponding airbag(s) are to be inflated, and/or a duration the corresponding airbags(s) are to be deflated), level of pressure (e.g., pounds per square inch or “psi”), duration of massage operation, a vibrational pattern, level of frequency, duration of vibrational operation, heating level, and duration of heating operation.

In some cases, the functions of voice recognition engine 110 may be performed by control engine 108. In such aspects, control engine 108 may receive the audio data from microphone 112. Moreover, control engine 108 may determine one or more words, phrases or terms, spoken by the user based on the audio data, as similarly described with voice recognition engine 110 (e.g., using one or more AI/ML models to determine one or more words, phrases or terms spoken by the user from the audio data).

For each matched word, phrase and/or term, control engine 108 may identify one or more corresponding operations (e.g., massage operations, heating operations and/or vibrational operations), or one or more corresponding operations of a mode of massage eye massager system 100 may perform, based on the command data. Moreover, control engine 108 may cause corresponding components of the determined operations, or mode of massage to perform or implement such operations. Additionally, or alternatively, for each matched word, phrase and/or term, control engine 108 may identify one or more corresponding adjustments to operations or operations of a mode of massage eye massager system 100 may be performing, based on the command data. Further, control engine 108 may cause corresponding components of the determined adjustments to perform or implement such adjustments.

As illustrated in FIG. 1, control engine 108 may be communicatively coupled to massage component(s) 102, heating component(s) 104 and/or vibration component(s) 106. In some cases, control engine 108 may identify or determine corresponding parameters or settings for each of the determined or identified operations based on the command data. Moreover, control engine 108 may identify one or more components associated with the identified operations and/or corresponding parameters and settings. Further, control engine 108 may electrically or electronically communicate with each of the identified components, via the one or more processors of eye massager 100, to cause each of the identified components to perform or implement the identified operation, mode of massage or adjustment in accordance with the corresponding identified parameters and settings (e.g., increase or decrease electrical power to the components in accordance with the identified parameters and settings).

In cases where the phrase, term and/or word included in the command data is associated with a mode of massage, the mode of massage may include one or more or a set of predetermined massage operations, heating operations, and/or vibrational operations. In such cases, the command data may identify, for such phrase, term and/or word, one or more or a set of massage settings, one or more or a set of heating settings and/or one or more or a set of vibrational settings.

For example, the term data may include the phrase “mode one.” Control engine 108 may access database 111 to determine the phrase “mode one” is included in command data stored in database 111. Moreover, control engine 108 may determine the phrase “mode one” is associated with a vibrational operation, a heating operation and a massage operation based on the command data. Based on the command data, control engine 108 may determine or identify the massage settings (e.g., a particular inflation and deflation pattern, a particular pressure level, and a particular duration) of the massage operation, the vibrational settings (e.g., a particular frequency pattern, a particular frequency level, and a particular duration) of the vibrational operation, and the heating settings (e.g., a heating level and a particular duration) of the heating operation. Based on the command data, control engine 18 may determine one or more massage components 102 associated with the massage settings, one or more heat components 104 associated with the heating settings, and one or more vibration components 106 associated with the vibrational settings. Further, control engine 108 may electrically or electronically communicate, via the one or more processors of eye massager 100, with the determined massage components 102 to perform the determined massage operations in accordance with the massage settings, with the determined heat components 104 to perform the determined heating operations in accordance with the heat settings, and with the determined vibration components 106 to perform the determined vibrational operations in accordance with the vibrational settings.

In some cases, the phrase, term and/or word may be associated with changing or switching a mode of massage eye massager system 100 may be performing or implementing. In such cases, eye massager system 100 may be configured to implement or perform a number of modes of massages. As described herein, each mode of massage may vary in settings related to massage operations, vibration operations and/or heating operations. Further each mode of massage may be associated with a set of health benefits, such as for tension relief or eye strain relief.

For example, eye massager system 100 may be configured to perform a first mode of massage in accordance with massage settings, vibrational settings and/or heating settings associated with the first mode. Moreover, eye massager system 100 may receive, from a user, an audio signal of the phrase “mode two” and voice recognition engine 110 may generate term data including the phrase “term two.” Based on the term data, control engine 108 may access database 111 to determine the phrase “mode two” is included in command data stored in database 111. Moreover, control engine 108 may determine the phrase “mode two” is associated with a vibrational operation, a heating operation and a massage operation and corresponding set of vibrational settings, heating settings and massage settings, based on the term data and the command data. In such an example, the set of vibrational settings, heating settings and massage settings of the phrase “mode two” may be different from the set of vibrational settings, heating settings and massage settings of the first mode. Based on the set of massage settings, vibrational settings and heating settings of the phrase “mode two” and the command data, control engine 108 may determine one or more massage components 102 associated with the massage settings, one or more heating components 104 associated with the heating settings and one or more vibration components 106 associated with the vibration settings. Moreover, control engine 108 may electrically or electronically communicate with the determined massage components 102, via the one or more processors of eye massager 100, to cause each of the determined massage components 102 to perform or implement one or more massage operations in accordance with the determined massage settings instead of the massage settings of the first mode. Further, control engine 108 may electrically or electronically communicate with the determined heating components 104, via the one or more processors of eye massager 100, to cause each of the determined heating components 104 to perform or implement one or more heating operations in accordance with the determined heating settings instead of the heat settings of the first mode. Moreover, control engine 108 may electrically or electronically communicate with the determined vibration components 106, via the one or more processors of eye massager 100, to cause each of the determined vibration components 106 to perform or implement one or more vibration operations in accordance with the determined vibration settings instead of the vibration settings of the first mode.

In some cases, the phrase, term and/or word included in the command data may be associated with one or more adjustments to one or more parameters or settings of a mode of massage, a massage operation, heating operation and/or vibrational operation that eye massager system 100 may be performing. In some aspects, the phrase, term and/or word may be associated with adjusting a duration of a mode of massage or a massage operation, heating operation and/or vibrational operation that eye massager system 100 may be performing. Examples of terms or phrases to adjust a duration of a mode of mode of massage, massage operation, heating operation and/or vibrational operation include, but are not limited to, “change time” followed by an amount of time, such as “change Time, 20 minutes.”

For example, eye massager system 100 may be configured to perform a first mode of massage in accordance with massage settings, vibrational settings and/or heating settings associated with the first mode of massage. In such an example, the massage settings of the first mode of massage may include a parameter or setting associated with a 10-minute duration. Moreover, eye massager system 100 may receive, from a user, an audio signal of the phrase “Change Time, 15 minutes” and voice recognition engine 110 may generate term data including the phrase “Change Time, 15 minutes.” Based on the term data, control engine 108, such as the control engine, may access database 111 to determine the phrase “Change Time, 15 minutes” is included in command data stored in database 111. Moreover, control engine 108 may determine the phrase “Change Time, 15 minutes” is associated with adjusting a duration of the massage operation of the first mode of massage to 15 minutes based on the term data and the command data. Based on the command data, control engine 108 may determine one or more massage components 102 may be associated with the phrase “Change Time, 15 Minutes.” Further, control engine 108 may electrically or electronically communicate with each of the massage components 102, via the one or more processors of eye massager 100, to cause the massage components 102 to perform or implement the adjustment—from a 10-15-minute duration.

In some aspects, the phrase, term and/or word may be associated with adjusting a pressure level (e.g., psi) of a massage operation eye massager system 100 may be performing. As described herein, each pressure level may be associated with one or a range of pressure levels. Examples of words, terms and/or phrases to adjust a pressure level provided by one or more airbags of eye massager system 100 may include, “No pressure,” “Soft Pressure,” “Medium Pressure,” and “Strong Pressure.” Each pressure level may be associated with one or a range of pressure levels.

For example, eye massager system 100 may be configured to perform a first mode of massage in accordance with a set of massage settings. In such an example, the set of massage settings may include a massage setting associated with a first pressure level. Moreover, eye massager system 100 may receive, from a user, an audio signal of the phrase “Medium Pressure” and voice recognition engine 110 may generate term data including the phrase “Medium Pressure.” “Medium Pressure” may have a pressure level that is different from the first pressure level. Based on the term data, control engine 108 may access database 111 to determine the phrase “Medium Pressure” is included in command data stored in database 111. Moreover, control engine 108 may determine the phrase “Medium Pressure” is associated with a massage operation and a massage setting of a particular pressure level based on the term data and the command data. Based on the command data, control engine 108 may identify one or more massage components 102 associated with the phrase “Medium Pressure.” Further, control engine 108 may electrically or electronically communicate with each of the massage components 102, via the one or more processors of eye massager 100, to cause the massage components 102 to perform or implement the massage setting associated with the phrase “Medium Pressure” instead of the massage setting associated with the first level of pressure.

In some aspects, the phrase, term and/or word may be associated with adjusting a heating level or temperature of a heating operation eye massager system 100 may be performing. Examples of words, terms and/or phrases to adjust a temperature level provided by a heat component 104 of eye massager system 100, such a heating device (e.g., copper coils) may include, “No Temperature,” “Low Heat,” “Medium Heat,” and “High Heat.” Each temperature level may be associated with one or a range of temperature levels. For example, low heat may have a lower temperature level than medium heat, medium heat may have a lower temperature level than high heat, and high heat has a higher temperature level than low heat and medium heat.

For example, eye massager system 100 may be configured to perform a first mode of massage in accordance with a set of heat settings. In such an example, the set of heat settings may include a heat setting associated with a first temperature/heat level. Moreover, eye massager system 100 may receive, from a user, an audio signal of the phrase “High Heat” and voice recognition engine 110 may generate term data including the phrase “High Heat.” Based on the term data, control engine 108, such as the control engine, may access database 111 to determine the phrase “High Heat” is included in command data stored in database 111. Moreover, control engine 108 may determine the phrase “High Heat” is associated with a heating operation and a heat setting of a particular temperature level based on the term data and the command data. “High Heat” may have a temperature level that is different from the first temperature level. Based on the command data, control engine 108 may identify one or more heat components 104 associated with the phrase “High Heat.” Further, control engine 108 may electrically or electronically communicate with each of heat components 104, via the one or more processors of eye massager 100, to cause heat components 104 to perform or implement the heat setting associated with the phrase “High Heat” instead of the heat setting associated with the first mode of massage.

In some aspects, the phrase, term and/or word may be associated with adjusting a vibrational level, frequency, or pattern of a vibrational operation eye massager system 100 may be performing. Examples of words, terms and/or phrases to adjust a frequency or vibrational level provided by vibration components 106 of eye massager system 100, such a motor device, may include, “Vibration Off,” “Low Vibration,” “Medium Vibration,” and “High Vibration.” Each vibration level may be associated with one or a range of vibration levels or frequency level.

For example, eye massager system 100 may be configured to perform a first mode of massage in accordance with a set of vibrational settings. In such an example, the set of vibrational settings may include a vibration setting associated with a first vibration level. Moreover, eye massager system 100 may receive, from a user, an audio signal of the phrase “Vibration Off” and voice recognition engine 110 may generate term data including the phrase “Vibration Off.” Based on the term data, control engine 108 may access database 111 to determine the phrase “Vibration Off” is included in command data stored in database 111. Moreover, control engine 108 may determine the phrase “Vibration Off” is associated with a vibrational operation and a vibration setting where the vibration ceases or the motor providing the vibration is off. Based on the command data, control engine 108 may identify one or more vibration components 106 associated with the phrase “Vibration Off.” Further, control engine 108 may electrically or electronically communicate with each of the vibration components 106, via the one or more processors of eye massager 100, to cause the vibration components 106 to perform or implement the vibration setting associated with the phrase “Vibration Off” (e.g., turning off the vibration components 106, such as turning off a motor).

In some cases, one or more phrases, terms and/or words spoken by a user may indicate one or more operations (e.g., massage operations, heating operations and/or vibrational operations) for a mode of massage, and corresponding one or more parameters or settings (as described herein as a “customized mode of massage”). In such cases, eye massager system 100 may receive, from a user, an audio signal indicating a parameter or setting of a massage operation (e.g., a pressure level), a parameter or setting of a heating operation (e.g., a heating level or temperature level), and/or a parameter or setting of a vibrational operation (e.g., a vibrational level). Based on the audio signal, eye massager system 100 may perform a customized mode of massage in accordance with the indicated the parameter or setting of the massage operation, parameter or setting of the heating operation, and/or the parameter or setting of the vibrational operation.

For example, eye massager system 100 may receive, from a user, an audio signal including the phrases “high pressure,” “medium heat,” and “vibration off.” Voice recognition engine 110 may generate term data including the phrases “high pressure,” “medium heat,” and “vibration off.” Based on the term data and command data, control engine 108 may access database 111 to determine the phrases “high pressure,” “medium heat,” and “vibration off” are included in command data stored in database 111. Based on the command data, control engine 108 may determine the phrase “high pressure” is associated with a massage operation. Moreover, control engine 108 may determine the massage operation may be associated with a massage setting of a particular pressure level, and one or more massage components 102, based on the term data. Based on the command data, control engine 108 may determine the phrase “medium heat” is associated with a heating operation. Moreover, control engine 108 may determine the heating operation may be associated with a heat or temperature setting indicating a particular temperature level, and one or more heating components 104, based on the term data. Based on the command data, control engine 108 may determine the phrase “Vibration Off” is associated with a vibrational operation and a vibration setting where the vibration ceases or the motor providing the vibration is off. Moreover, the control engine 108 may determine the vibrational operation may be associated with one or more vibration components 106. Further, control engine 108 may electrically or electronically communicate with each of the determined massage components 102, heating components 104 and vibration components 106, via the one or more processors of eye massager 100, to cause the determined massage components 102, heating components 104 and vibration components 106 to perform or implement the corresponding massage setting, heat setting, and vibration setting, respectively.

In some aspects, eye massager system 100 may generate and store customized massage data in database 111. As described herein, the customized massage data may identify the one or more operations (e.g., massage operations, heating operations and/or vibrational operations) of a “customized mode of massage,” and corresponding parameters or settings. In such aspects, control engine 108 may generate customized massage data based on the operations and corresponding settings and parameters determined from associated term data). In some instances, control engine 108 may store the customized massage data within the command data.

For example, eye massager system 100 may receive, from a user, an audio signal including the phrases “medium pressure,” “high heat,” and “vibration on.” Voice recognition engine 110 may generate term data including the phrases “medium pressure,” “high heat,” and “vibration on.” Based on the term data and command data, control engine 108 may access database 111 to determine the phrases “medium pressure,” “high heat,” and “vibration on” are included in command data stored in database 111. Based on the command data, control engine 108 may determine the phrase “medium pressure” is associated with a massage operation. Moreover, control engine 108 may determine the massage operation may be associated with a massage setting of a particular pressure level, and one or more massage components 102, based on the term data. Based on the command data, control engine 108 may determine the phrase “high heat” is associated with a heating operation. Moreover, control engine 108 may determine the heating operation may be associated with a heat or temperature setting indicating a particular temperature level, and one or more heating components 104, based on the term data. Based on the command data, control engine 108 may determine the phrase “Vibration on” is associated with a vibrational operation and a vibration setting where the vibration is performed or the motor providing the vibration is on. Moreover, the control engine 108 may determine the vibrational operation may be associated with one or more vibration components 106. Further, control engine 108 may generate customized massage data identifying the determined massage operation, heating operation, and vibrational operation, and corresponding determined parameters and settings.

In some instances, control engine 108 may perform or implement the operations of the customized mode of massage when eye massager system 100 is powered up or turned on. In some cases, the customized massage data may be associated with a particular term, phrase or word. In such cases, the command data may include the particular term along with the customized massage data. Moreover, a user may cause eye massager system 100 to perform the operation(s) included in the customized mode of massage upon speaking the particular term, phrase or word. For example, database 111 may store command data including customized massage data and corresponding phrase, term and/or word, such as “customized massage.” Moreover, eye massager system 100 may receive, from a user, an audio signal including the phrases “customized massage.” Further, eye massager system 100 may perform any of the processes described herein to implement the operation(s) of the customized massage data (e.g., the massage operations, heating operations and/or vibrational operations indicated in customized massage data).

In some cases, a phrase, term and/or word included in the command data may cause eye massager system 100 to shut down. Examples of words, terms and/or phrases to shut down eye massager system 100 may include “turn off.” For example, while eye massager system 100 is operating, eye massager system 100 may receive, from a user, an audio signal of the phrase “turn off” and voice recognition engine 110 may generate term data including the phrase “turn off.” Based on the term data, control engine 108 may access database 111 to determine the phrase “turn off” is included in command data stored in database 111. Moreover, control engine 108 may determine the phrase “turn off” is associated with shutting down eye massager system 100. Based on the determined operation the control engine may, via the one or more processors of eye massager system 100, initiate a sequence of electrical processes to shut down eye massager system 100. In some instances, after eye massager system 100 is shut down, a user may power up eye massager system 100 manually, such as via a power up button (e.g., a power up button included in user interface module 113).

In some cases, voice recognition engine 110 and/or control engine 108 may be dormant or inactive until a particular predetermined phrase, term or word is spoken by a user operating eye massager system 100. In such cases, one or more processors of eye massager system 100 may execute a wake-up engine that is operatively or communicatively coupled to microphone 112. Moreover, the wake-up engine may generate a signal or instruction to activate a control engine 108 and/or voice recognition engine 110 that are dormant in response to determining an audio signal of the particular predetermined phrase, term or word, such as a wake-up term or phrase, is detected or obtained by the microphone. In some instances, command data may include the particular predetermined phrase, terms or words, such as “Hey Eye Massager.” In such instances, the wake-up engine may access database 111 to determine whether an audio signal includes the particular predetermined phrase, term or word.

For example, while voice recognition engine 110 and/or control engine 108 may be dormant or inactive, the wake-up engine may be actively listening, via microphone 112. Moreover, microphone 112 may detect and obtain audio signals from a user and generate audio data based on the obtained audio signals. Further, the microphone may transmit or provide the audio data to the wake-up engine. The wake-up engine may determine one or more words, phrases or terms, spoken by the user based on the audio data. In some cases, the wake-up engine, similarly described with voice recognition engine 110 and/or control engine 108, may apply one or more AI/ML models, such as a speech recognition-based AI/ML model, to the audio data. Based on the application of the one or more AI/ML models to the audio data, the wake-up engine may perform any of the described examples processes to determine one or more words, phrase or terms spoken by the user. Moreover, the wake-up engine may generate term data including the determined words, phrases or terms. Further, the wake-up module may access database 111 to determine whether the term data includes the wake-up term or phrase. In examples where the wake-up engine determines the term data includes the wake-up term or phrase, the wake-up engine may implement one or more processes to activate the dormant or inactive voice recognition engine 110 and/or the control engine 108.

In some instances, voice recognition engine 110 may be partially dormant and may perform any of the above-described processes with regard to the wake-up engine to listen for the wake-up term or phrase. In such instances, voice recognition engine 110 may fully activate itself and/or control engine 108 upon determining the wake-up term or phrase was detected by microphone 112 of eye massager system 100.

In some instances, voice recognition engine 110 and/or control engine 108 may be temporarily activated for a predetermined period of time, such as 1 minute. Moreover, upon the predetermined period of time lapsing and voice recognition engine 110 and/or control engine 108 is activated and does not detect or determine a user has spoken a phrase, term and/or word associated with a particular mode or an adjustment to one or more settings of a mode, massage operation, heating operation and/or a vibrational operation, voice recognition engine 110 and/or control engine 108 may go inactive or dormant again. Eye massager system 100, such as wake-up engine or partially dormant voice recognition engine 110, may activate voice recognition engine 110 and/or control engine 108 again upon detecting or determining a user spoke a wake-up term or phrase.

In some cases, a phrase, term and/or word included in the command data may be a profile term. The profile term may be associated with a profile of a user and a mode of massage, such as a preferred mode of massage indicated by the user. In some aspects, the user operating eye massager system 100 may register the profile term and indicate the preferred mode of massage to be associated with the profile term. In some instances, the user may use a mobile computing device, such as a smartphone, laptop or desktop, to register the profile term and indicate the preferred mode of massage associated with the profile term. In such instances, the user may use a microphone of the mobile computing device to register the profile term. The mobile computing device may determine one or more words, terms or phrases that comprise the profile term. Moreover, the user may use one or more input devices of the mobile computing device to indicate a mode of massage to associate with the profile term. The mobile computing device may generate profile data characterizing the profile term, the indicated mode of massage, the settings of a massage operation, heating operation and/or vibrational operation associated with the indicated mode of massage and corresponding components. Further, the mobile computing device may transmit to eye massager system 100 the profile data, via communication interface 115. Control engine 108 may access database 11 to include the profile data in the command data. In some aspects, the mobile computing device may communicate with eye massager system 100 via communication interface 115 and over one or more networks, such as, but not limited to wired and/or wireless intranet, extranet, Internet, cellular, Bluetooth, infrared, and/or any other short range, long range, local, regional, global communications mechanism, means, approach, protocol and/or network, as well as any combination(s) thereof

In some cases, the user may use eye massager system 100 to register the profile term and indicate the preferred mode of massage associated with the profile term. In such instances, the user may use microphone 112 to register the profile term. Voice recognition engine 110 may obtain the audio data of the profile term and determine one or more words, terms or phrases that comprise the profile term. Moreover, voice recognition engine 110 may generate profile term data and provide the profile term data to control engine 108. Further, the user may use one or more input devices of user interface module 113 to indicate a mode of massage to associate with the profile term. Control engine 108 may generate profile data that includes the one or more words, terms and/or phrases that comprise the profile term, the indicated mode of massage, the settings of a massage operation, heating operation and/or vibrational operation associated with the indicated mode of massage and corresponding components. Further, control engine 108 may store the profile data in database 111. In some instances, control engine 108 may include the profile data in the command data.

Moreover, control engine 108 may automatically cause eye massager system 100 to implement the mode of massage (e.g., one or more of the massage operation, heating operation and/or vibrational operation associated with the mode of massage) associated with the profile of the user when determining the user spoke the profile term.

For example, microphone 112 may receive or obtain, from a user, an audio signal of the phrase “massage me” and voice recognition engine 110 may generate term data including the phrase “massage me.” Based on the term data, control engine 108 may access database 111 to determine the phrase “massage me” is a profile term included in command data stored in database 111. Moreover, control engine 108 may determine the phrase “massage me” is associated with a particular mode of massage based on the command data. Further, control engine 108 may determine the vibrational operation, a heating operation and a massage operation and corresponding set of vibrational settings, heating settings and massage settings, based on the command data. Based on the set of massage settings, vibrational settings and heating settings of the phrase “massage me” and the command data, control engine 108 may determine one or more massage components 102 associated with the massage settings, one or more heating components 104 associated with the heating settings and one or more vibration components 106 associated with the vibration settings. Moreover, control engine 108 may electrically or electronically communicate with the determined massage components 102, via the one or more processors of eye massager 100, to cause each of the determined massage components 102 to perform or implement one or more massage operations in accordance with the determined massage settings. Further, control engine 108 may electrically or electronically communicate with the determined heating components 104, via the one or more processors of eye massager 100, to cause each of the determined heating components 104 to perform or implement one or more heating operations in accordance with the determined heating settings. Moreover, control engine 108 may electrically or electronically communicate with the determined vibration components 106, via the one or more processors of eye massager 100, to cause each of the determined vibration components 106 to perform or implement one or more vibration operations in accordance with the determined vibration settings.

In some aspects, the mobile computing device and/or voice recognition engine 110 may determine one or more characteristics or features of the voice of the user (e.g., pitch, tone and cadence). In such aspects, control engine 108 may obtain data including information related to the one or more characteristics of the voice of the user from the mobile computing device and/or voice recognition engine 110. Moreover, control engine 108 may store such data with the registered profile term. Further, control engine 108 may use such data when determining whether the user has spoken the profile term.

For example, microphone 112 may receive or obtain, from a user, an audio signal of the phrase “relaxation time.” Based on the audio signal, voice recognition engine 110 may determine one or more characteristics or features of the voice of the user, such as the pitch, cadence and tone as well as the one or more words, terms and/or phrases included in the audio signal of the phrase “relaxation time.” Moreover, voice recognition engine may generate term data that includes the phrase “relaxation time” along with the associated features and characteristics of the voice of the user when the user spoke the phrase “relaxation time.” Based on the term data, control engine 108 may access database 111 to whether determine the phrase “relaxation time” is a profile term included in command data stored in database 111. Moreover, control engine 108 may access database 111 to whether determine the features and characteristics included in the term data match or are included with the profile term included in the command data. In examples where control engine 108 determines both the phrase of the term data is a profile term included in the command data and the features and characteristics of the voice of the user of the term data are a match to the characteristics and features of a voice associated with the profile term in the command data, control engine 108 may perform any of the above describe processes to implement the massage operations, heating operations and/or vibrational operations associated with the profile term.

As illustrated in FIG. 1, eye massager system 100 may include user interface module 113. User interface module 113 may include a user interface that enables a user to select a mode of massage, change to a different mode of massage, adjust one or more parameters or settings of a mode of massage, massage operation, heating operation and/or vibrational operation, an audio operation (e.g., turn off or on one or more speakers of eye massager system 100, change an audio item being outputted by eye massager system 100 via the speakers, and increase/decrease the volume) and/or a power operation (e.g., power up or power down eye massager system 100). In some examples, the user interface may include one or more input devices, such as, but not limited to, buttons (e.g., capacitive or mechanical), switches, toggles, keypad, keyboard, and touch screen (e.g., pressure-sensitive touch screen). Each of the input devices may be associated with an operation eye massager system 100 may perform, such as a heating operation, vibrational operation, massage operation, audio operations or power operations.

In some cases, the input devices may be mechanical button. Additionally, the user interface module 113 may include a material that covers the mechanical buttons. That way, a user may still interact with the mechanical buttons and the cover may prevent the elements, such as water, from coming into contact with the mechanical buttons. In some instances, the material may be formed out of any material, such as silicon, leather, rubber, etc.

In some instances, the user interface may include an input device associated with a mode of massage. As described herein, the mode of massage may include one or more or a set of massage operations, one or more or a set of heating operations, and/or one or more or a set of vibrational operations. In some cases, the input device may be configured to select and/or change a mode of massage eye massager system 100 is to perform. In some aspects the input device may be configured to be time dependent. For instance, the input device may be configured to cycle through each mode of massage eye massager system 100 may perform each time a user interacts with the input device for a predetermined time interval (e.g., the user presses down the button for at least 7 seconds before the next mode of massage is presented).

For example, the input device may be configured to cycle through each mode of massage eye massager system 100 may perform each time a user interacts with the input device (e.g., press down the button). Based on the mode of massage the user selects, the user interface may generate a signal or data indicating the mode of massage the user selected. The user interface may transmit the signal or data to control engine 108. Control engine 108 may perform any of the described processes to determine a set of massage operations, heating operations, and/or vibrational operations associated with the selected mode of massage and cause corresponding components (e.g., massage component(s) 102, heating component(s) 104, and/or vibration component(s) 106) to implement or perform the determined set of massage operations, heating operations, and/or vibrational operations, respectively.

In some instances, the user interface may include an input device associated with adjusting a setting or parameter of a massage operation, such as the pressure level. In some cases, the input device may be configured to select and/or change a setting of the massage operation performed by eye massager system 100. In some aspects the input device may be configured to be time dependent. For instance, the input device may be configured to cycle through each setting of the massage operation each time a user interacts with the input device for a predetermined time interval (e.g., the user presses down the button for at least 3 seconds before the next setting of the massage operation is presented and presented).

For example, the input device may be configured to cycle through multiple pressure level settings for the massage operation each time a user interacts with the input device (e.g., press down the button). Based on the pressure level setting the user selects, the user interface may generate a signal or data indicating the pressure level setting the user selected. The user interface may transmit the signal or data to control engine 108. Control engine 108 may perform any of the described processes to determine the pressure level setting the user selected and cause corresponding components (e.g., massage component(s) 102) to implement or perform the selected pressure level setting.

In some instances, the user interface may include an input device associated with adjusting a setting or parameter of a vibrational operation, such as the vibration level. In some cases, the input device may be configured to select and/or change a setting of the vibrational operation performed by eye massager system 100. In some aspects the input device may be configured to be time dependent. For instance, the input device may be configured to cycle through each setting of the vibrational operation each time a user interacts with the input device for a predetermined time interval (e.g., the user presses down the button for at least 7 seconds before the next setting of the vibrational operation is presented and presented).

For example, the input device may be configured to cycle through multiple vibrational level settings for the vibrational operation each time a user interacts with the input device (e.g., press down the button). Based on the vibrational level settings the user selects, the user interface may generate a signal or data indicating the vibrational level settings the user selected. The user interface may transmit the signal or data to control engine 108. Control engine 108 may perform any of the described processes to determine the vibrational level settings the user selected and cause corresponding components (e.g., vibration component(s) 106) to implement or perform the selected vibrational level setting.

In some instances, the user interface may include an input device associated with adjusting a setting or parameter of a heating operation, such as the temperature. In some cases, the input device may be configured to select and/or change a setting of the heating operation performed by eye massager system 100. In some aspects the input device may be configured to be time dependent. For instance, the input device may be configured to cycle through each setting of the heating operation each time a user interacts with the input device for a predetermined time interval (e.g., the user presses down the button for at least 10 seconds before the next setting of the heating operation is presented and presented).

For example, the input device may be configured to cycle through multiple temperature settings for the heating operation each time a user interacts with the input device (e.g., press down the button). Based on the temperature setting the user selects, the user interface may generate a signal or data indicating the temperature setting the user selected. The user interface may transmit the signal or data to control engine 108. Control engine 108 may perform any of the described processes to determine the temperature setting the user selected and cause corresponding components (e.g., heat component(s) 104) to implement or perform the selected temperature setting.

In some aspects, an input device may be configured to select or change settings of multiple types of operations (e.g., massage operations, heating operations and/or vibrational operations). In such aspects, the input device may be configured to select or present different settings of different types of operations based on the interaction between the user and the input device. For instance, the input device may be configured to select, change or present different settings of a massage operation when a user interacts with (e.g., presses down) the input device for more than 7 seconds. Moreover, the input device may be configured to select, change or present different settings of a vibrational operation when a user interacts with (e.g., presses down) the input device for at most 3 seconds.

In some cases, an input device may be configured to select or change a mode of massage eye massager system 100 may perform and settings of one or more types of operations (e.g., massage operations, heating operations and/or vibrational operations). For instance, the input device may be configured to select, change or present different settings of a massage operation when a user interacts with (e.g., presses down) the input device for at most 3 seconds. Moreover, the input device may be configured to select, change or present different modes of massages eye massager system 100 may perform when a user interacts (e.g., presses down) the input device for more than 7 seconds.

In some instances, the user interface may include an input device associated with powering up or powering down eye massager system 100. For instance, while eye massager system 100 is powered on, a user may power down the eye massager system 100 when the user interacts with the device (e.g., presses down) for more than 10 seconds. Moreover, while eye massager system 100 is powered down, a user may power up the eye massager system 100 when the user interacts with the device (e.g., presses down) for at least 3 seconds.

In some cases, eye massager system 100 may output audio, such as music, podcasts, or any other audio item. In such cases, eye massager system 100 may include one or more speakers. The speakers may output audio of an audio item. In some instances, microphone 112 may not detect output from the speakers. In such instances, the speakers may be positioned 8-15 cm so that microphone 112 may not pick up an output from the speakers. Moreover, database 111 may store audio data including one or more audio items, such as music. Further, a user may cause eye massager system 100 to output music stored in database 111 based on phrases, words and/or terms spoken by the user. In some aspects, a phrase, term and/or word included in the command data may be associated with selecting an audio item to play, playing an audio item, changing an audio item currently playing, increasing the volume of an audio item being outputted by the speakers of the eye massager system 100, decreasing the volume of an audio item being outputted by the speakers of the eye massager system 100, turning off the speakers and turning on the speakers. Examples of such terms, words or phrases, may include, but are not limited to, “play music,” “change music,” “increase volume,” “decrease volume,” “sound off,” and “sound on.” In some instances, the audio item may be played while a mode of massage, massage operation, heating operation and/or vibrational operation is being performed by eye massager system 100. Alternatively, the audio item may be played when the eye massager system 100 is not implementing or performing a mode of massage, massage operation, heating operation and/or vibrational operation.

For example, eye massager system 100 may be configured to output a first audio item through one or more speakers of eye massager system 100. Moreover, eye massager system 100 may receive, from a user, an audio signal of the phrase “change music” and voice recognition engine 110 may generate term data including the phrase “change music.” Based on the term data, control engine 108 may access database 111 to determine the phrase “change music” is included in command data stored in database 111. Moreover, control engine 108 may determine the phrase “change music” is associated with the operation of selecting and outputting a different or next audio item. Based on the phrase “change music” and/or the determined associated operation, control engine 108 may access database 111 to select another audio item. Based on the selected audio item, control engine 108 may output the selected audio item through the speakers of eye massager system 100.

In another example, eye massager system 100 may be configured to output a first audio item through one or more speakers of eye massager system 100. Moreover, eye massager system 100 may receive, from a user, an audio signal of the phrase “increase volume” and voice recognition engine 110 may generate term data including the phrase “increase volume.” Based on the term data, control engine 108 may access database 111 to determine the phrase “increase volume” is included in command data stored in database 111. Moreover, control engine 108 may determine the phrase “increase volume” is associated with the operation of increase the volume or loudness of the first audio item outputting through the speakers of eye massager system 100. Based on the phrase “increase volume” and/or the determined associated operation, control engine 108 may communicate with the speakers of eye massager system 100 to increase the volume or loudness.

In another example, eye massager system 100 may be configured to output a first audio item through one or more speakers of eye massager system 100. Moreover, eye massager system 100 may receive, from a user, an audio signal of the phrase “decrease volume” and voice recognition engine 110 may generate term data including the phrase “decrease volume.” Based on the term data, control engine 108 may access database 111 to determine the phrase “decrease volume” is included in command data stored in database 111. Moreover, control engine 108 may determine the phrase “decrease volume” is associated with the operation of decreasing the volume or loudness of the first audio item outputting through the speakers of eye massager system 100. Based on the phrase “decrease volume” and/or the determined associated operation, control engine 108 may communicate with the speakers of eye massager system 100 to decrease the volume or loudness.

In yet another example, eye massager system 100 may be configured to output a first audio item through one or more speakers of eye massager system 100. Moreover, eye massager system 100 may receive, from a user, an audio signal of the phrase “sound off” and voice recognition engine 110 may generate term data including the phrase “sound off.” Based on the term data, control engine 108 may access database 111 to determine the phrase “sound off” is included in command data stored in database 111. Moreover, control engine 108 may determine the phrase “sound off” is associated with the operation of powering down or turning off the speakers of eye massager system 100. Based on the phrase “sound off” and/or the determined associated operation, control engine 108 may communicate with the speakers of eye massager system 100 to power down or turn off.

Alternatively, in examples where the speakers of eye massager system 100 are already powered down or turned off, term data including the phrase “sound on” may power up or turn on the speakers. For example, based on the term data including the phrase “sound on,” control engine 108 may access database 111 to determine the phrase “sound on” is included in command data stored in database 111. Moreover, control engine 108 may determine the phrase “sound on” is associated with the operation of powering up or turning on the speakers of eye massager system 100. Based on the phrase “sound on” and/or the determined associated operation, control engine 108 may communicate with the speakers of eye massager system 100 to power up or turn on.

In some aspects, an audio item may be played from another mobile computing device, such as a smartphone, laptop, desktop, etc. In such aspects, the other mobile computing device may communicate and interact with eye massager system 100 through communication interface 115. Moreover, eye massager system 100 may, by implementing one or more processes and operations similarly described above with respect to phrases, terms and/or words spoken by a user. For instance, a user may, through eye massager system 100, select an audio item to play, play an audio item, and change an audio item currently being played based on a phrase, word and/or term spoken by the user. In such an instance, the audio item to be outputted by the speakers of eye massager system 100 may be stored or played from the mobile computing device. Moreover, while the mobile computing device is communicating with eye massager system 100, and based on the term data, control engine 108 may communicate and/or access the mobile computing device (instead of database 111) to select or play an audio item stored or played from the mobile computing device or to change the audio item currently being played to another audio item stored or to be played from the mobile computing device. In some aspects, the mobile computing device may communicate with eye massager system 100 via communication interface 115 and over one or more networks, such as, but not limited to wired and/or wireless intranet, extranet, Internet, cellular, Bluetooth, infrared, and/or any other short range, long range, local, regional, global communications mechanism, means, approach, protocol and/or network, as well as any combination(s) thereof.

In some examples, eye massager system 100 may initiate the massage operation(s) or the mode of massage based on sensor data generated by the sensing component(s) 116 of eye massager system 100. Additionally, or alternatively, eye massager system 100 may adjust the parameter(s) or setting(s) of the massage operation(s) or the mode of massage currently being performed by eye massager system 100 based on the sensor data. Examples of sensing component(s) 116, include, but are not limited to, heart rate sensors, pulse oximeter sensors, photoplethysmogram (PPG) sensor, temperature sensors, electrodermal activity (EDA) sensors, and other sensors that generate sensor data associated with biometric information of a user. In some instances, sensing component(s) 116 may interface with one or more portions of a head of a user using eye massager system 100.

In some aspects, control engine 108 may be communicatively coupled to sensing component(s) 116. Control engine 108 may obtain sensor data associated with a user from sensing component 116. Based on the sensor data, control engine 108 may determine biometric information of the user. Examples of biometric information that control engine 108 may determine may include, but is not limited to, heart rate, blood oxygen levels, blood pressure, respiration, other cardiovascular variability assessments, skin conductance or electrodermal activity, and facial temperature. Based on the biometric information of the user and a corresponding predetermined threshold criteria, such as a value or one or more ranges of values, control engine 108 may determine a state of the user (e.g., a stressed state, a relaxed state or a stress level). For example, based on the heart rate of the user and a predetermined threshold heart rate, control engine 108 may determine whether the user is stressed or relaxed (e.g., a heart rate above or equal to a predetermined threshold heart rate may indicate the user is in a stressed state, while a heart rate below the predetermined threshold heart rate may indicate the user is a relaxed state). In another example, based on the determined skin conductivity (e.g., caused by perspiration or other physical changes) and a set predetermined ranges of conductivity, control engine 108 may determine which range of conductivity the determined skin conductivity is associated with or falls within. Each range of conductivity may be associated with a stress level. Control engine 108 may determine a stress level associated with the range of conductivity the determined skin conductivity is associated with or falls within.

Based on the state of the user, control engine 108 may determine one or more operations (e.g., massage operation(s), heating operation(s) and/or vibration operation(s) or a mode of massage eye massager system 100 may implement or perform and corresponding parameter(s) or setting(s) based on the state of the user. For example, control engine 108 may obtain sensor data from one or more temperature sensors. Control engine 108 may determine a temperature of the user based on the sensor data. Based on the determined temperature and one or more predetermined ranges of temperatures, control engine 108 may determine a range of temperatures the determined is associated with or falls within. Each of the predetermined ranges of temperatures may be associated with a stress level. Control engine 108 may determine a stress level associated with the range of temperatures the determined is associated with or falls within. Control engine 108 may determine one or more operations or a mode of massage eye massager system 100 may implement or perform and corresponding parameter(s) or setting(s) based on the determined stress level.

For instance, if the determined range of temperatures is associated with a high level of stress, control engine 108 may determine one or more operations or a mode of massage corresponding to the high level of stress, such as massage operation(s) associated with a lower level of air pressure, heating operation(s) associated with a lower level of heat or temperature, and/or vibration operation(s) with a lower level of vibration. Conversely, if the determined range of temperatures is associated with a low level of stress, control engine 108 may determine one or more operations or a mode of massage corresponding to the low level of stress, such as massage operation(s) associated with a higher level of air pressure, heating operation(s) associated with a higher level of heat or temperature, and/or vibration operation(s) with a higher level of vibration. As described herein, control engine 108 may electrically or electronically communicate with massage component(s) 102, heating component(s) 104 and/or vibration component(s) 106, via the one or more processors of eye massager 100, to cause massage component(s) 102, heating component(s) 104 and/or vibration component(s) 106 to perform or implement the determined operation(s) or mode of massage and corresponding parameter(s) or setting(s).

Additionally, or alternatively, control engine 108 may determine adjustments to parameter(s) or setting(s) of a mode of massage, massage operation(s), heating operation(s) and/or vibration operation(s) based on the state of the user. For example, while a user is being massaged by one or more massage components 102, one or more heating components 104 and/or one or more vibration components 106, control engine 108 may obtain sensor data from one or more PPG sensors. Control engine 108 may determine a heart rate of the user based on the sensor data. Based on the determined heart rate and a predetermined threshold heart rate, control engine 108 may determine whether a user is experiencing a high level of stress (e.g., whether the determined heart rate is greater than or equal to the predetermined threshold heart rate (e.g., a value)). Control engine 108 may determine one or more adjustments to parameter(s) or setting(s) of a mode of massage, massage operation(s), heating operation(s) and/or vibration operation(s).

For instance, if the determined heart rate is equal to or greater than a predetermined threshold heart rate (e.g., a value), control engine 108 may determine one or more adjustments to parameter(s) or setting(s) of a mode of massage, massage operation(s), heating operation(s) and/or vibration operation(s), such as adjustments associated with lowering a level of air pressure, a level of heat or temperature, and/or a level of vibration. As described herein, control engine 108 may electrically or electronically communicate with massage component(s) 102, heating component(s) 104 and/or vibration component(s) 106, via the one or more processors of eye massager 100, to cause massage component(s) 102, heating component(s) 104 and/or vibration component(s) 106 to perform or implement the determined adjustment(s).

In some examples, sensing components 116 may enable control engine 108 to adjust the pressure level of the airbag(s) or compartment(s) of massage components 102. In such examples, sensing components 116 may include sensing components, such as, pressure sensors, that generate sensor data indicating a pressure level (e.g., a value corresponding to a pressure level) between a portion of a head or face of a user and the airbag(s) or compartment(s). In some aspects, control engine 108, via such sensing components, may “sense” when the airbags or compartments of massage components 102 make contact with a face of a user and prevent the airbags or compartments from providing too much pressure to the face of the user. Control engine 108 may determine whether the pressure provided by the airbag(s) or compartment(s) is too much pressure to the face of the user based on a predetermined threshold criteria (e.g., a value corresponding to a maximum pressure level eye massager system 100 may provide to the user during one or more massage operations). Such sensing components may generate sensor data indicating a pressure level (e.g., a value corresponding to a pressure level). The sensing components may provide the sensor data to control engine 108. Based on the sensor data, control engine 108 may determine the pressure level (e.g., psi, kpa, etc.) being provided to a head or face of the user or being exerted by the airbags or compartments. Control engine 108 may control one or more massage components 102, such as a motor or air pump motor providing air to or removing air from the airbags or compartments, to prevent the airbags or compartments from providing pressure above the predetermined threshold criteria, such as a predetermined threshold pressure level.

For example, the user may wear an eye massager device or apparatus including eye massager system 100. Moreover, eye massager system 100 may include such sensing components. As control engine 108 implements any of the processes described herein to inflate the airbags or compartments of eye massager system 100, such sensing components may detect the pressure levels being provided by or exerted from the airbags or compartments onto the face of the user. Further, such sensing components may generate sensor data and provide the sensor data to control engine 108. Control engine 108 may determine and monitor the pressure levels being provided by or exerted from the airbags or compartments onto the face of the user based on the sensor data. In instances where control engine 108 determines the pressure levels are equal to or above a predetermined pressure level, control engine 108 may implement processes to stop or prevent the motor or air pump motor from providing additional air to the airbags or compartments. For instance, control engine 108 may electrically or electronically communicate, via the one or more processors of eye massager system 100, with the motor or air pump motor to stop providing air to the airbags or compartments. Otherwise, control engine 108 may not interfere with or may continue to implement processes that cause motor or air pump motor from providing additional air to the airbags or compartments. For instance, control engine 108 may electrically communicate or electronically, via the one or more processors of eye massager system 100, with the motor or air pump motor to continue providing air to the airbags or compartments.

In some cases, control engine 108, via such sensing components, may “sense” when the airbags or compartments of massage component(s) 102 make contact with a face of a user and may increase the pressure provided by the airbag(s) or compartment(s) to the head or face of the user. Control engine 108 may determine whether the pressure level provided by the airbag(s) or compartment(s) to the face of the user is too little based on a predetermined criteria threshold (e.g., a value corresponding to a pressure level eye massager system 100 may provide to the user during one or more massage operations). Such sensing components may generate sensor data indicating a pressure level (e.g., a value corresponding to a pressure level). The sensing components may provide the sensor data to control engine 108. Based on the sensor data, control engine 108 may determine the pressure level (e.g., psi, kpa, etc.) being provided to a head or face of the user or being exerted by the airbags or compartments. Based on the determined pressure level being below the predetermined threshold criteria, such as a predetermined threshold level, control engine 108 may control massage component(s) 102, such as a motor or air pump motor, to add air to the airbag(s) or compartment(s) of massage component(s) 102 until the determined pressure level at least equals or meets the predetermined threshold criteria.

In some aspects, control engine 108 may adjust the pressure level of the airbag(s) or compartment(s) of massage component(s) based on multiple predetermined threshold criteria, such as predetermined threshold pressure levels. As described herein, each of the multiple predetermined threshold criteria may enable control engine 108 to determine whether the pressure level provided by the airbag(s) or compartment(s) to one or more portions of a head of a user is too little or too much. Control engine 108 may cause one or more massage components 102, such as a motor or air pump motor, to remove, stop providing, or provide air to the airbag(s) or compartment(s) of massage component(s) 102 based on whether the pressure level provided by the airbag(s) or compartment(s) to one or more portions of a head of a user is too little or too much.

In some instances, eye massager system 100 may be configured to perform multiple predetermined massage operations and each of the predetermined massage operations may be associated with a predetermined pressure level. For instance, eye massager system 100 may be configured to perform three predetermined massage operations. A first massage operation of the three predetermined massage operations may be associated with a predetermined pressure level of 0 kPa, a second massage operation of the three predetermined massage operations may be associated with a predetermined pressure level of 25 kPa, and a third massage operation of the three predetermined massage operations may be associated with a predetermined pressure level of 36 kPa. In some examples, each predetermined pressure level may serve as a threshold criterion used by control engine 108 to adjust the pressure level of the airbag(s) or compartment(s) of massage components 102 applied to portion(s) of a user's head. For instance, eye massager system 100, such as control engine 108, may perform a first massage operation and the first massage operation may be associated with a predetermined pressure level (e.g., 25 kPa). While performing the first massage operation, such sensing components (e.g., one or more pressure sensors) may generate sensor data indicating the pressure level provided by airbag(s) or compartment(s) of massage components 102 to portion(s) of a head of a user. Based on the sensor data, control engine 108 may determine the actual pressure level being provided. Control engine 108 may compare the determined pressure level to the predetermined pressure level associated with the first massage operation (the predetermined threshold criterion). If the determined pressure level is too low (e.g., below the predetermined pressure level), control engine 108 may control massage component(s) 102, such as an air pump motor, to add air to the airbag(s) or compartment(s) until the determined pressure level meets or exceeds the predetermined pressure threshold level. Conversely, if the pressure level is too high (e.g., above the predetermined pressure level), control engine 108 may control massage component(s) 102 to prevent additional air or release air from the airbags or compartments until the determined pressure level meets or equals the predetermined pressure level.

In some instances, such sensing components may be positioned on the exterior of one or more of the airbags or compartments of eye massager system 100. In some cases, such sensing components may be positioned within or a part of tubing, or one or more hoses or connectors that couple the motor or air pump motor of eye massager system 100 and the airbags or compartments. For instance, eye massager system 100 may include a printed circuit board (PCB) that includes one or more processors of eye massager system 100, As described herein the one or more processors may execute control engine 108 and voice recognition engine 110. Further, the PCB may include one or more massage components 102, such as a motor or air pump and/or tubing that connects the motor or air pump to the airbag(s) or compartment(s) of massage component(s) 102. Such sensing components may be coupled to the PCB and a portion of the tubing to generate sensor data indicating a pressure level within the tubing. Examples of such sensing components that eye massager system 100 may include, but are not limited to, are capacitive pressure sensors, piezoelectric pressure sensors, piezoresistive pressure sensors, Microelectroechanical System (MEMS) pressure sensors, optical pressure sensors, and pressure transducers.

In some cases, eye massager system 100 may provide state information associated with eye massager system 100. As described herein, the state information may be associated with a biometric information associated with a user using or operating eye massager system 100 (e.g., heart rate, skin conductance or electrodermal activity, and facial temperature), state of the user (e.g., stressed, relax, asleep, a level of stress, etc.), one or more massage operations or a mode of massage that eye massager system 100 may be performing or implementing (e.g., a mode of massage and/or massage operation(s) eye massager system 100 is implementing or performing and/or corresponding parameters, such as a massage duration or time remaining, a massage pressure level, a vibrational frequency level, a heating level or temperature, etc.), and/or one or more corresponding adjustments to parameter(s) or setting(s) of the massage operation(s), heating operation(s), vibration operation(s), and/or a mode of massage that eye massager system 100 may be performing. In some examples, control engine 108 may provide the state information to display system 117. Display system 117 may display the state information.

In some instances, display system 117 may include a display device. Examples of a display device display system 117 may include, but are not limited to, a light emitting diode (LED) dot matrix or matrix panel, a liquid-crystal display (LCD) screen, an LED screen and an organic LED (OLED) screen. The display device and/or display system 117 may display or present the state information, via one or more formats. Examples of formats the display device and/or display system 117 may present the state information, includes, but is not limited to, color coded visual indicators (e.g., green light indicates a relaxed or calm state, red light indicates elevated stress, blue light indicates a state of deep relaxation or sleep, a particular color light associated with a particular operation that eye massager system 100 may be performing, such as a green light for low pressure level (or “soft pressure”) massage operation(s), a red light for high pressure level (or “strong pressure”) massage operation(s), a yellow light for medium pressure level (or “medium pressure”) massage operations, etc.), graphics/images, such as emoticons or symbols (e.g., a smiling face may indicate relaxation while a frowning face or exclamation mark may indicate stress or tension), textual output (e.g., alphanumeric or textual descriptions corresponding to the state of the user, such as “relaxed” to indicate a low-stress state or “stressed” to indicate a user is in a stressed state), and animations/videos (e.g., videos or animations correlating to the state of the user, such as a wave-like animation to indicate the user is relaxed, a flashing red animation to indicate the user is stressed or has elevated stress levels, videos or animations unrelated to the user, such as the time of the year (e.g., a video or animation related to the current season), etc.).

In some examples, control engine 108 may determine the state information associated with eye massager system 100 based on one or more parameter(s) or setting(s) of massage operation(s), heating operation(s) and/or vibration operation(s) (e.g., a set of massage settings, heat settings and/or vibrational settings, respectively, or one or more adjustments to the set of massage settings, heat settings and/or vibrational settings, respectively) determined for eye massager system 100. For example, based on term data associated with one or more words or phrases spoken by a user and command data, control engine 108 may determine one or more massage settings (e.g., a particular inflation and deflation pattern, a particular pressure level, and a particular duration), vibrational settings (e.g., a particular frequency pattern, a particular frequency level, and a particular duration), and/or heating settings (e.g., a heating level and a particular duration). Control engine 108 may provide the determined massage setting(s), vibrational setting(s) and/or heating setting(s) to a display device of display system 117. The display device may output, display or present information associated with the determined massage setting(s), vibrational setting(s) and/or heating setting(s). As described herein, control engine 108 may electrically or electronically communicate with massage component(s) 102, heating component(s) 104 and/or vibration component(s) 106, via the one or more processors of eye massager 100, to cause massage component(s) 102, heating component(s) 104 and/or vibration component(s) 106 to perform or implement the determined massage setting(s), vibrational setting(s) and/or heating setting(s).

In another example, based on term data associated with one or more words or phrases spoken by a user and command data, control engine 108 may determine one or more adjustments to the massage setting(s), vibrational setting(s) and/or heating setting(s). Control engine 108 may provide the determined adjustment(s) to a display device of display system 117. The display device may output, display or present information associated with the determined adjustment(s). As described herein, control engine 108 may electrically or electronically communicate with massage component(s) 102, heating component(s) 104 and/or vibration component(s) 106, via the one or more processors of eye massager 100, to cause massage component(s) 102, heating component(s) 104 and/or vibration component(s) 106 to perform or implement the determined adjustment(s).

In another example, control engine 108 may obtain sensor data from one or more heart rate sensors. Control engine 108 may determine a heart rate of the user based on the sensor data. Based on the determined heart rate and a predetermined threshold heart rate (e.g., a value), control engine 108 may determine the determined heart rate is greater than or equal to the predetermined threshold heart rate. Based on such determinations, control engine 108 may determine one or more parameters or settings of a mode of massage, massage operation, heating operation and/or vibration operation (e.g., massage setting(s), vibrational setting(s) and/or heating setting(s)). Control engine 108 may provide the determined parameters or setting to a display device of display system 117. The display device may output, display or present information associated with the determined parameters or settings. As described herein, control engine 108 may electrically or electronically communicate with massage component(s) 102, heating component(s) 104 and/or vibration component(s) 106, via the one or more processors of eye massager 100, to cause massage component(s) 102, heating component(s) 104 and/or vibration component(s) 106 to perform or implement the determined parameters or settings.

In another example, while a user is being massaged by one or more massage components 102, one or more heating components 104 and/or one or more vibration components 106, control engine 108 may obtain sensor data from one or more heart rate sensors. Control engine 108 may determine a heart rate of the user based on the sensor data. Based on the determined heart rate and a predetermined threshold heart rate (e.g., a value), control engine 108 may determine the determined heart rate is greater than or equal to the predetermined threshold heart rate. Based on such determinations, control engine 108 may determine one or more adjustments to a parameter or setting of a mode of massage, massage operation, heating operation and/or vibration operation, such as adjustments associated with lowering a level of air pressure, a level of heat or temperature, and/or a level of vibration. Control engine 108 may provide the determined adjustment(s) to a display device of display system 117. The display device may output, display or present information associated with the determined adjustment(s). As described herein, control engine 108 may electrically or electronically communicate with massage component(s) 102, heating component(s) 104 and/or vibration component(s) 106, via the one or more processors of eye massager 100, to cause massage component(s) 102, heating component(s) 104 and/or vibration component(s) 106 to perform or implement the determined adjustment(s).

In some aspects, control engine 108 may determine the state information associated with eye massager system 100 based on biometric information associated with a user associated with eye massager system 100 (e.g., heart rate, skin conductance or electrodermal activity, and facial temperature). For example, sensing component(s) 116 may generate sensor data associated with a user associated with eye massager system 100. Based on the sensor data, control engine 108 may determine biometric information associated with the user, as described herein. Control engine 108 may provide the determined biometric information to a display device of display system 117. The display device may output, display or present the biometric information.

In some examples, control engine 108 may determine the state information associated with eye massager system 100 based on a state of a user associated with eye massager system 100 (e.g., stressed, relax, asleep, a level of stress, etc.). As described herein, control engine 108 may determine the state of the user based on biometric information of a user associated with eye massager system 100 and a corresponding predetermined threshold criteria. For example, control engine 108 may determine a heart rate of the user based on sensor data generated by one or more heart rate sensors. Based on the heart rate of the user and a set of predetermined ranges of heart rates, control engine 108 may determine a range of heart rates the heart rate of the user is associated with or falls within. Each set of predetermined ranges of heart rates may be associated with a stress level. Control engine 108 may determine a stress level associated with the range of heart rates the determined heart rate of the user is associated in or falls within. Control engine 108 may provide the determined state of the user to a display device of display system 117. The display device may output, display or present the information associated with the state of the user.

As illustrated in FIG. 1, eye massager system 100 may include power source 114, such as one or more batteries. Power source 114 may store electrical power. Moreover, the batteries of power source 114 may supply or provide power to one or more hardware components of eye massager system 100, such as, but not limited to, one or more processors of eye massager system 100, a memory device of eye massager system 100, one or more components of massage components 102 (e.g., an air pump or motor), one or more components of heat components 104 (e.g., the heating device), one or more components of vibration components 106 (e.g., a motor), microphone 112, communication interface 115, one or more sensing components 116, and/or display system 117. For instance, power source 114 may provide power to the hardware components via one or more voltage rails. Examples of the batteries includes, but is not limited to, alkaline batteries, nickel metal hydride batteries, and lithium-ion batteries. In some instances, the one or more batteries may be rechargeable.

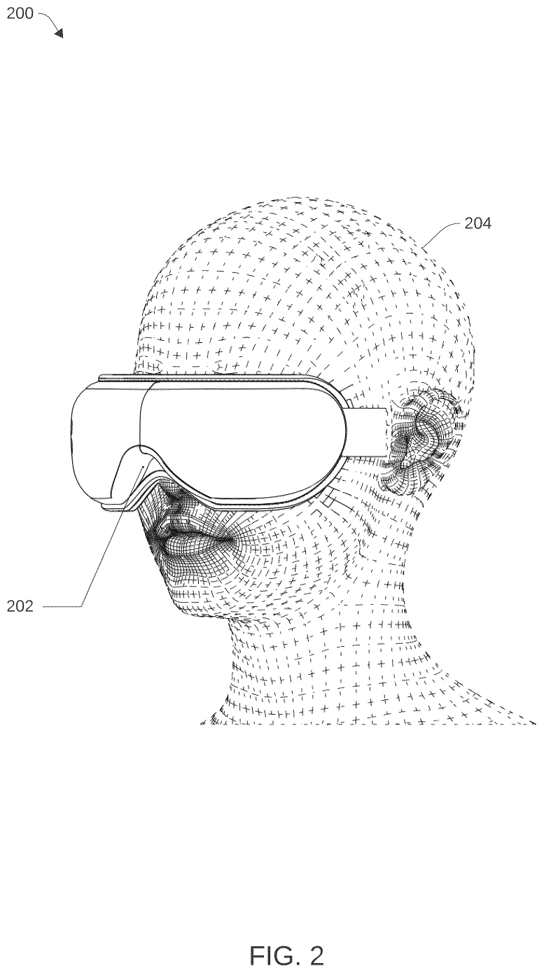

As described herein, eye massager system 100 may be worn by a user when the user uses eye massager system 100. FIG. 2 illustrates how a user may wear eye massager system 100. As illustrated in FIG. 2, eye massager 202 may be placed on and worn by a user, such as user 204. Moreover, eye massager 202 may include one or more components, such as hardware components, and/or software systems, as similarly described with eye massager system 100.

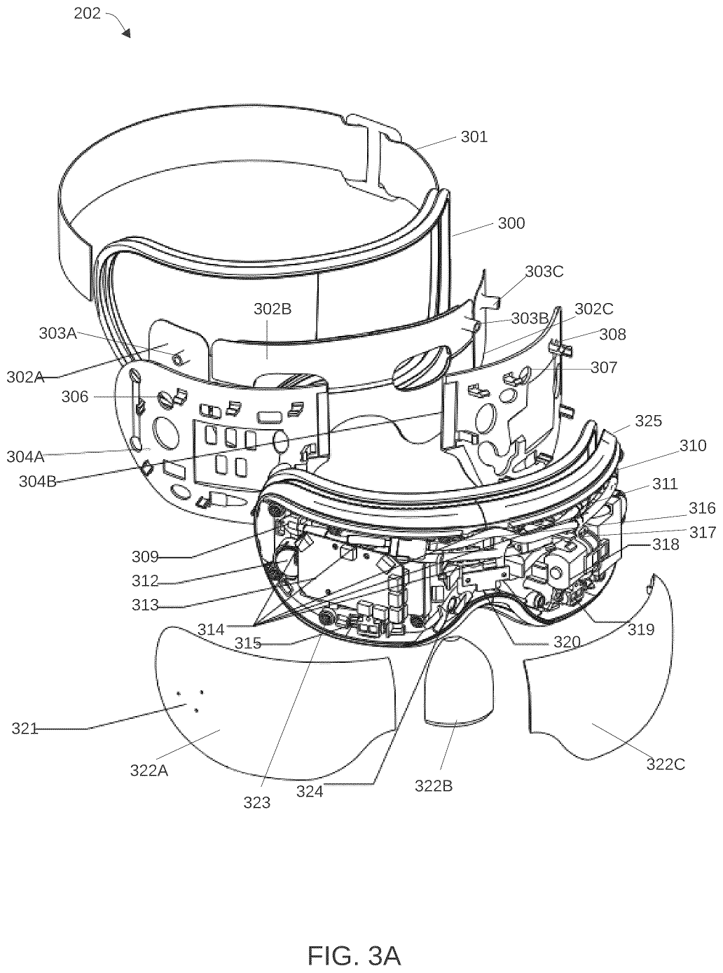

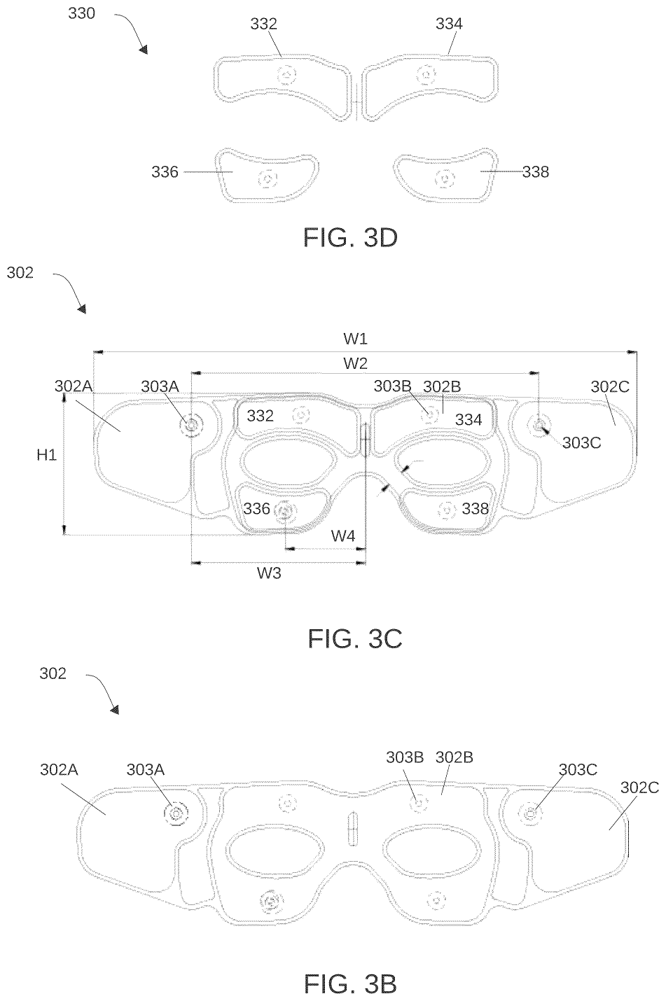

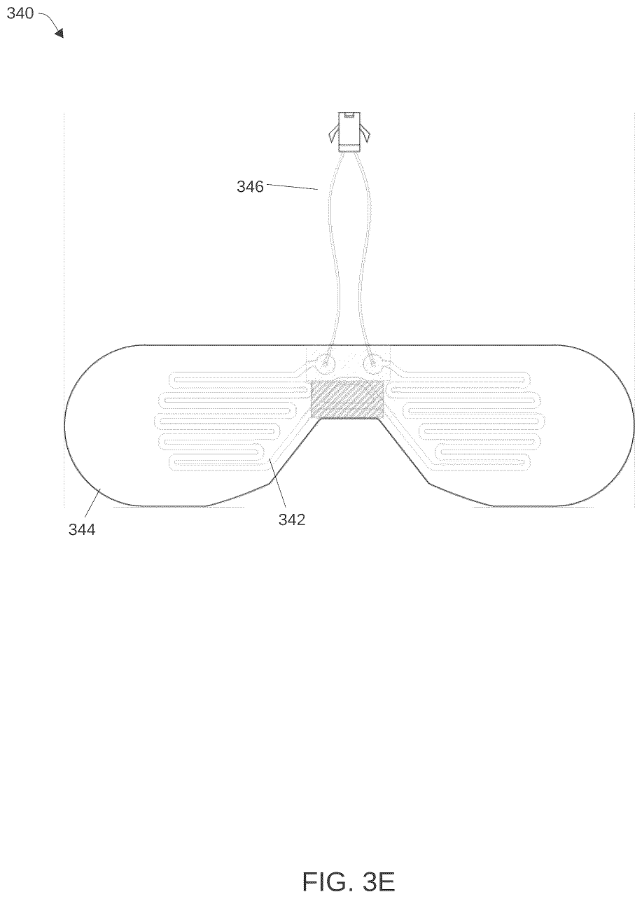

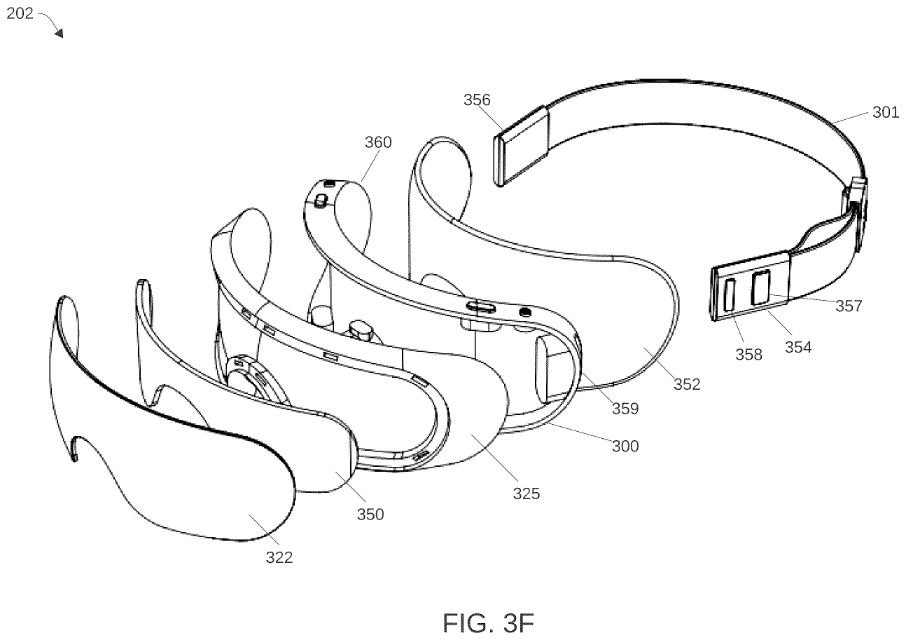

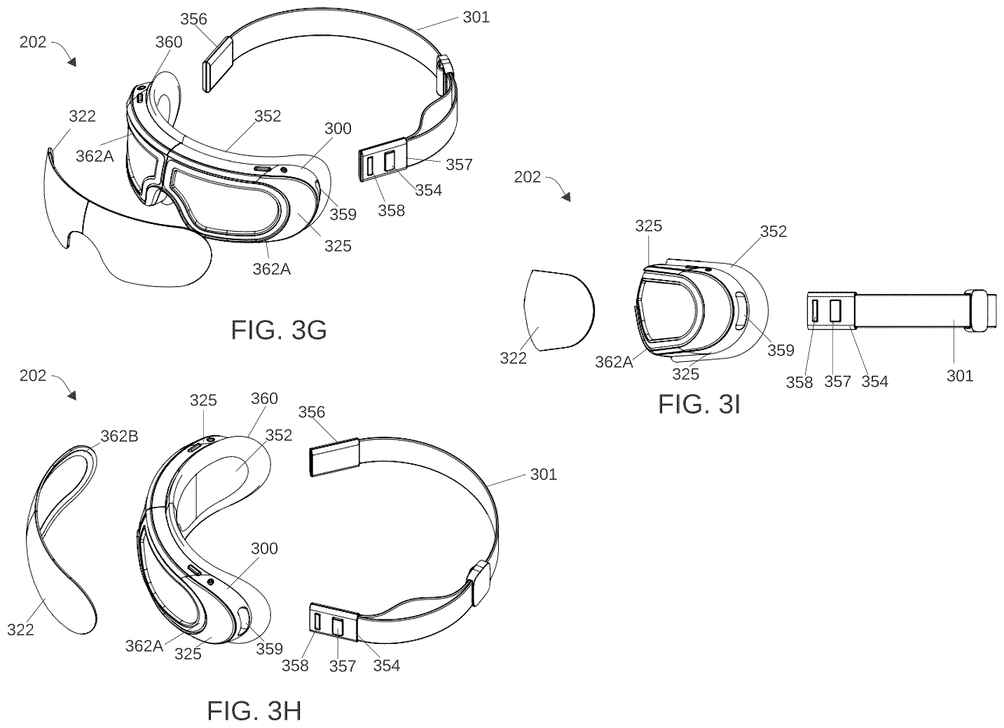

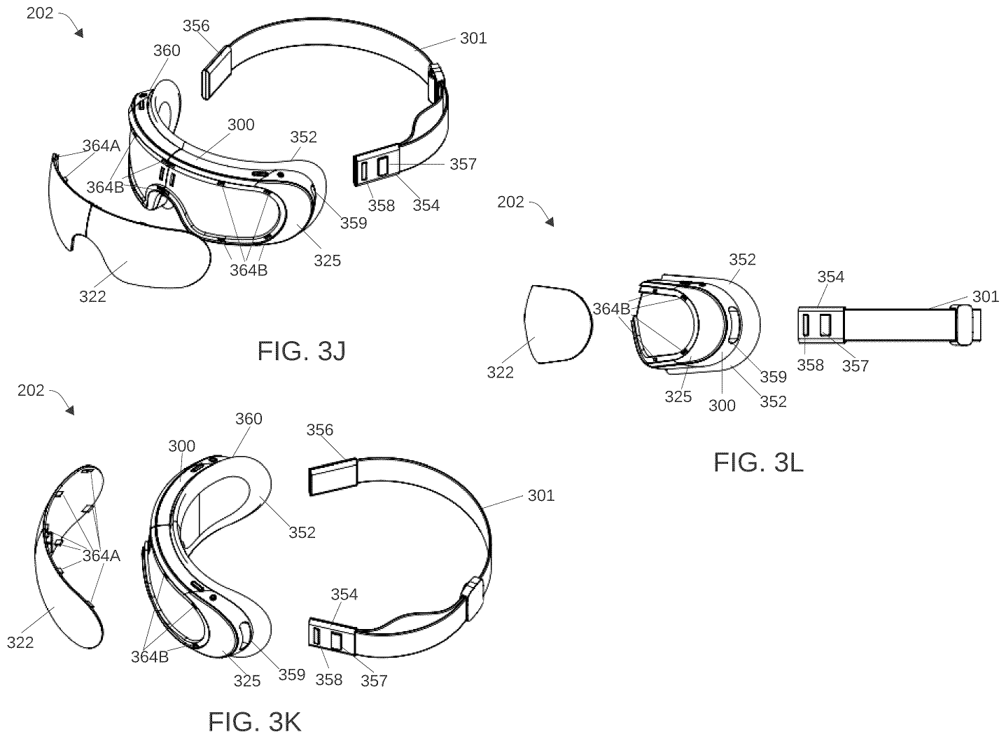

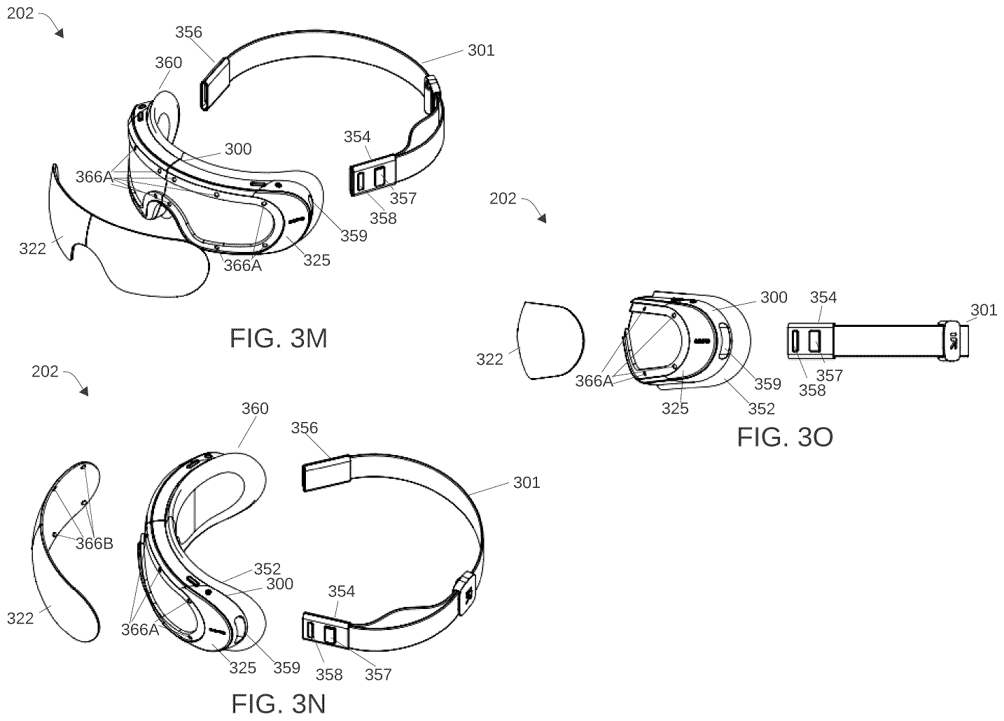

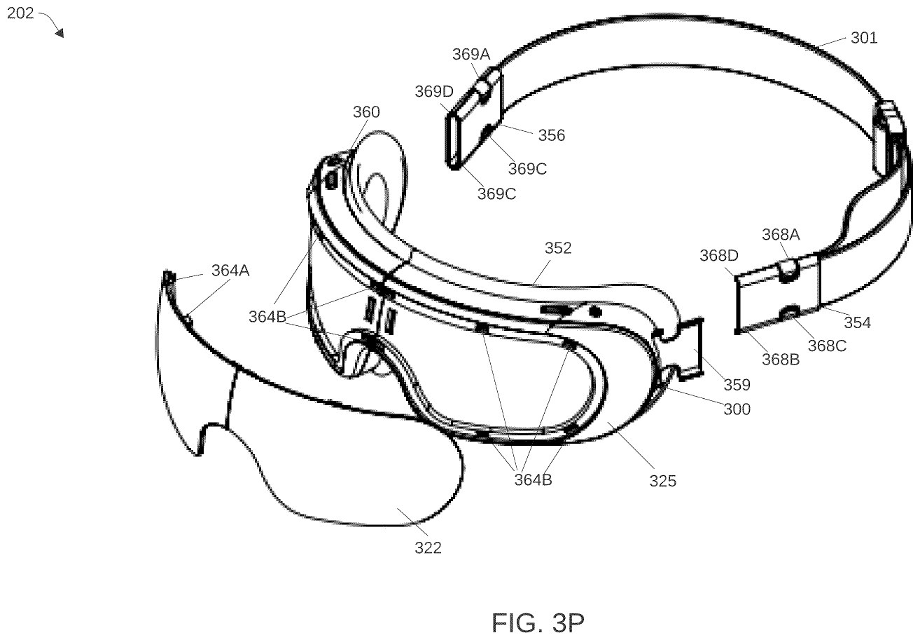

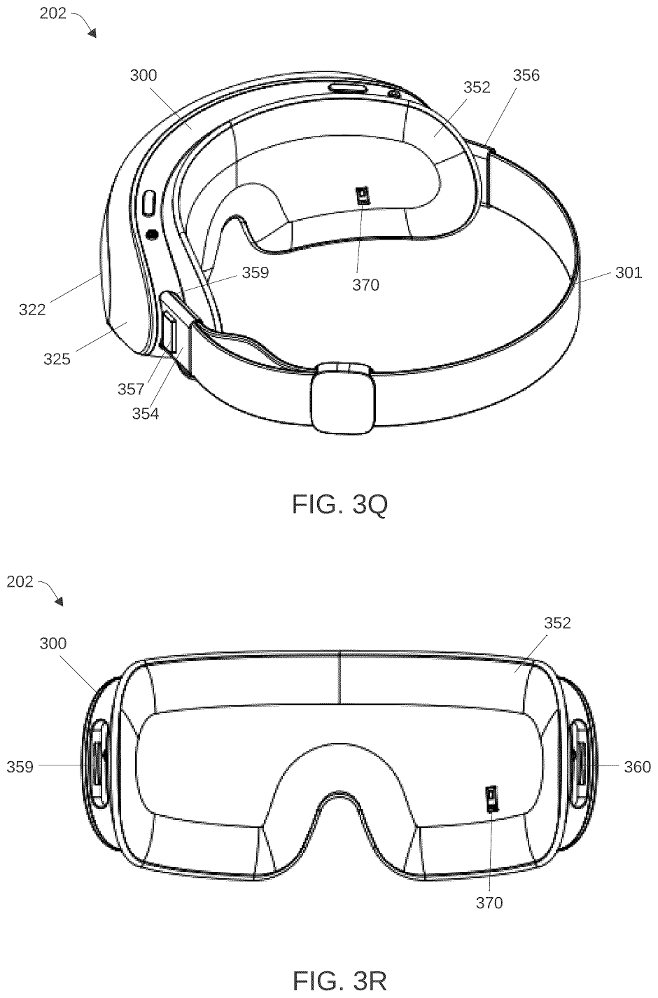

FIG. 3A illustrates an exploded view of the components included in eye massager 202. As illustrated in FIG. 3A, eye massager 202 may include one or more housing elements, such as housing element 300, housing element 304, housing element 322, and housing element 325. The housing elements provide structural support for eye massager 202 and hardware components included in eye massager 202. Moreover, eye massager 202 may include strap component 301. Strap component 301 may be formed out of a flexible material that used to secure eye massager 202 onto a user (e.g., user 204 of FIG. 2), such as leather, elastic material or spandex. In some instances, strap component 301 may include a fastener, a buckle, and/or a slide buckle.

As illustrated in FIG. 3A, eye massager 202 may include one or more compartments or airbags 302 (e.g., airbag 302A, airbag 302B, and airbag 302C), one or more nozzles (e.g., nozzle 309, nozzle 310, and nozzle 311), speaker 312, controller device 313, one or more tubing or hose (e.g., hose 314), one or more motors (e.g., vibration motor 315, vibration motor 319, and air pump motor 318), one or more valves (e.g., valve 316 and valve 317), electric charging connector 320, and one or more input devices 321. Although not illustrated in FIG. 3A, eye massager 202 may include power source components, one or more memory devices, a communication interface device, and heating device.

In some cases, housing element 325 may include one or more hardware components such as, but not limited to one or more nozzles (e.g., nozzle 309, nozzle 310, and nozzle 311), speaker 312, controller device 313, one or more tubing or hose (e.g., hose 314), one or more motors (e.g., vibration motor 315, vibration motor 319, and air pump motor 318), one or more valves (e.g., valve 316 and valve 317), electric charging connector 320, and power source components (not shown).

In some aspects, controller device 313 may include one or more processors of eye massager 202. As described herein, the one or more processors of eye massager 202 may execute a control engine and a voice recognition engine, as similarly described with control engine 108 and voice recognition engine 110 of FIG. 1. The control engine and the voice recognition may have the same functionalities and may perform the processes as described herein with control engine 108 and voice recognition engine 110, respectively. Further, the one or more processors of eye massager 202, similar to the one or more processors of eye massager system 100, may be electrically or communicatively coupled to speaker 312, one or more motors, such as vibration motor 315, vibration motor 319, and air pump motor 318, one or more valves (e.g., valve 316 and valve 317)—if electrical, one or more input devices 321, microphone 324, the power source components, one or more memory devices, a communication interface device and the heating device.

In some examples, controller device 313 may include a printed circuit board (PCB) and the PCB may include, but is not limited to, the one or more processors of eye massager 202, the communication interface device, and one or more memory device. In some instances, the functionalities, processes and/or operations, the communication interface device, and the one or more memory devices, may be similar to communication interface 115 and database 111, respectively.

In some cases, air pump motor 318 may be configured to inflate and deflate one or more compartments or airbags 302 of eye massager 202 (e.g., airbag 302A, airbag 302B, airbag 302C). As illustrated in FIG. 3A, air pump motor 318 may be coupled to one or more nozzles (e.g., nozzle 309, nozzle 310, and nozzle 311) via one or more tubing or hoses, such as hose 314. Each nozzle may be coupled to an airbag or compartment of eye massager 202 (e.g., airbag 302A, airbag 302B, airbag 302C). Moreover, the tubing or hoses may be connected to or include one or more valves, such as, valve 316 and valve 317. The valves may enable air pump motor 318 to selectively direct air to particular airbags 302 of eye massager 202 and to selectively remove air from particular airbags 302 of eye massager 202. In some instances, air pump motor 318 may include a device that reduces the sound of air pump motor 318 when operating, such as a silicon housing that fully or partially covers a body of the air pump motor 318.

In some aspects, the valves may be an electronic valve (e.g., an electromagnetic solenoid valve). In such cases, the one or more processors may electrically or electronically control the electronic valves to selectively inflate particular airbags 302 of eye massager 202. As described herein, each mode of massage or a massage operation may include massage settings or parameters that indicate a particular inflation/deflation pattern of the airbags 302. Based on such settings or parameters, the control engine may electrically or electronically control the electronic valves and air pump motor 318, via the one or more processors, to selectively direct air from air pump motor 318 to inflate particular airbags 302 of eye massager 202, and selectively remove air by air pump motor 318 to deflate particular airbags 302 of eye massager 202 in accordance with inflation/deflation pattern associated with such settings or parameters.

Moreover, each mode of massage or a massage operation may include massage settings or parameters that indicate a particular duration of the inflation/deflation pattern of the airbags 302. The control engine may electrically or electronically control the electronic valves and air pump motor 318, via the one or more processors, to selectively inflate/deflate the airbags 302 of eye massager 202 for a particular duration of time in accordance with corresponding massage settings. Further, each mode of massage or a massage operation may include massage settings or parameters that indicate a particular duration of the inflation/deflation pattern of the airbags 302. The control engine may electrically or electronically control the electronic valves, via the one or more processors, to selectively inflate the airbags 302 of eye massager 202 to a particular pressure level in accordance with associated massage settings. In some instances, air pump motor 318, nozzle 309, nozzle 310, nozzle 311, valve 316, and valve 317 may be similar in function and operation as described with the air pumps, nozzles, and valves of massage component(s) 102, respectively.

In some cases, the one or more vibrational motors, such as vibration motor 315 and vibrational motor 319, of eye massager 202 may be configured to vibrate. In some examples, the vibrational motors may be an electric motor with an off-centered weight attached to a rotational shaft of the motor. When in operation and when the rotational shaft rotates, such vibrational motors may vibrate. As described herein, each mode of massage or a vibration operation may include vibration settings or parameters. In some instances, the vibration settings or parameters may indicate a particular vibration level or frequency level. Based on such settings or parameters, the control engine may electrically or electronically control the vibrational motors, via the one or more processors, to vibrate at the vibrational level or frequency as indicated by such vibration settings.

In some examples, the vibration settings or parameters may indicate a particular duration of the vibrational operations. Based on such settings or parameters, the control engine may electrically or electronically control the vibrational motors, via the one or more processors, to vibrate for the duration indicated by such vibration settings.

In some instances, the vibration settings or parameters may indicate a particular pattern of vibration. Based on such settings or parameters, the control engine may electrically or electronically control the vibrational motors, via the one or more processors, to vibrate in accordance with the vibrational pattern indicated by such vibration settings.

In some aspects, speaker 312 may be configured to output an audio item. Similar to the one or more speakers of eye massager system 100, speaker 312 may output an audio item stored in eye massager 202 or from another mobile computing device. As described herein, the audio item may be stored in the one or more memory devices of eye massager 202 or may be provided by a mobile computing device. Moreover, the control engine of eye massager 202, similar to control engine 108, may communicate with a mobile computing device via the communication interface device of eye massager 202.

In some examples, microphone 323 may be configured to detect and obtain audio signals from a user. Microphone 323 may generate audio data that the voice recognition and the control engine of eye massager 202 may use to determine a mode of massage to implement or perform or adjustments to a parameter or setting of a mode of massage, massage operation, heating operation and/or vibration operation. As described herein, microphone 323 may have the same functionalities and may perform the same processes as described herein with microphone 112.

In some cases, the power source components of eye massager 202, similar to power source 114 of eye massager system 100, may include one or more batteries. Similar to power source 114, the batteries of eye massager 202 may supply or provide power to one or more processors of eye massager 202, one or more memory devices of eye massager 202, a communication interface device of eye massager 202, speaker 312, one or more motors (e.g., vibration motor 315, vibration motor 319, and air pump motor 318), one or more valves (e.g., valve 316 and valve 317), if electric, and one or more input devices 321. For instance, power source 114 may provide power to such hardware components via one or more voltage rails. Examples of the batteries includes, but is not limited to, alkaline batteries, nickel metal hydride batteries, and lithium-ion batteries. In some instances, the one or more batteries may be rechargeable. In such instances, and as illustrated in FIG. 3A, eye massager 202 may include electric charging connector 320. Electric charging connector 320 may be configured to receive a power from another power source (e.g., a charging cable connected to another power source, such as another battery or an outlet).

In some cases, housing element 322, including housing element 322A, housing element 322B, and housing element 322C, may couple to or attach to housing element 325. When coupled to or attached to housing element 325, housing element 322, may protect the hardware components housed in housing element 325 from the elements. In some aspects, housing element 322 may attach to housing element 325 by one or more snap fit elements, such as a cantilever type snap fit element (e.g., a cantilever beam and a tapered hook), an annular type snap fit element (e.g., a circular or ring-shaped protrusion component that couples to a corresponding groove or channel component) and/or a torsional type snap fit element. Additionally, or alternatively, housing element 322 may attach to housing element 325 by one or more coupling elements, such as a screw. Further, housing element 322 may house one or more input devices. As described herein, the one or more input devices of eye massager 202 may have the same functionalities and may perform the same processes as described herein with the input devices of user interface module 113. In some instances, housing element 322 may be formed from various materials, such as glass, metal, metal alloys ceramic, polymeric material, fabrics, and plastics (e.g., Acrylonitrile Butadiene Styrene (ABS), polycarbonate, acrylic or any other polymer plastic). In some examples, housing element 322 may be transparent, opaque, reflective on one side while the other side is transparent, or reflective on both sides.

In some cases, housing element 304, including housing element 304A and housing element 304B may couple to or attach to housing element 325. As illustrated in FIG. 3A, housing element 325 may include a back side and a front side. Moreover, housing element 304A and housing element 304B may each include a back side and a front side. Further, each of the front sides of the housing element 304A and housing element 304B may couple or attach to the back side of housing element 325, while housing element 322 attaches to the front side of housing element 325. In some aspects, housing element 304A and housing element 304B may attach to housing element 325 by one or more snap fit elements and/or one or more coupling elements.

Further, housing element 304A and housing element 304B may each include one or more airbag openings. For example, housing element 304A may have a thickness that defines an opening or airbag opening 306. Moreover, housing element 304A may have a thickness that defines a first opening or airbag opening 307 and a second opening or airbag opening 308. In some aspects, and when the front side of housing element 304A is coupled to the back side of housing element 325, airbag opening 306 may align with nozzle 309 and/or nozzle 309 may be partially or fully within or through airbag opening 306. Moreover, when the front side of housing element 304B is coupled to the back side of housing element 325, airbag opening 307 may align with nozzle 311 and/or nozzle 311 may be partially or fully within or through airbag opening 307, while airbag opening 308 may align with nozzle 310 and/or nozzle 310 may be partially or fully within or through airbag opening 308.

Further, housing element 304, including housing element 304A and housing element 304B may provide structural support for the airbags 302 of eye massager 202. For example, when eye massager 202 is worn by a user, such as user 204 of FIG. 2, the airbags 302, when inflated, may push off of housing element 304 and towards the face of the user, providing directed pressure in the form of a massage to one or more portions of a face of a user wearing eye massager 202, such as around the left eye, right eye, left temple, and/or right temple of the user. In some instances, housing element 304, including housing element 304A and housing element 304B may be formed from various materials, such as glass, ceramic, polymeric material of material, and plastic. In some aspects, housing element 304, including housing element 304A and 304B, may have a width of 11-13 cm and a height of 7-9 cm. In some cases, eye massager 202 may be wider to better cover the temples of a user. In such cases, eye massager 202 may have a width of 12-15 cm and a height of 7-9 cm.

As described herein, the airbags 302 of eye massager 202, such as airbag 302A, airbag 302B and airbag 302C may be configured to inflate and deflate to provide directed pressure in the form of a massage to one or more portions of a face of a user wearing eye massager 202, such as around the left eye (e.g., supraorbital of the left eye, infraorbital of the left eye, portions of the face between the orbital bone of the left eye near the bridge of the nose and/or portions of the face between the orbital bone of the left eye and the temple), around the right eye (e.g., supraorbital of the right eye, infraorbital of the right eye, portions of the face between the orbital bone of the right eye near the bridge of the nose and/or portions of the face between the orbital bone of the right eye and the temple), the left temple, and/or the right temple of the user (e.g., user 204 of FIG. 2). As illustrated in FIG. 3A, each of the airbags 302 may include an airbag nozzle, such as airbag nozzle 303A, airbag nozzle 303B and airbag nozzle 303C. The airbag nozzle of each airbag may couple to one of the nozzles of eye massager 202. For example, airbag nozzle 303A of airbag 302A may be inserted into airbag nozzle 309 through airbag opening 306 or airbag nozzle 309 may be inserted into airbag nozzle 303A through airbag opening 306, airbag nozzle 303B of airbag 302B may be inserted into nozzle 311 through airbag opening 307 or nozzle 311 may be inserted into airbag nozzle 303B through airbag opening 307, and/or airbag nozzle 303C of airbag 302C may be inserted into nozzle 310 through airbag opening 308 or nozzle 310 may be inserted into airbag nozzle 303C through airbag opening 308. That way air may be pumped int the respective airbag or removed from the respective airbag by air pump motor 318.

In some cases, the one or more airbags 302 may be configured or arranged in one or more layers. For example, and referring to FIG. 3B, the one or more airbags 302 may be configured or arranged in a single layer. As illustrated in FIG. 3B, the single layer arrangement of the one or more airbags 302 may include airbag 302A, airbag 302B and airbag 302C. In some aspects airbag 302A, airbag 302B and airbag 302C may be one compartment. Alternatively, airbag 302A, airbag 302B and airbag 302C may include multiple compartments.

In another example, and referring to FIG. 3C, the one or more airbags 302 may be configured or arranged in multiple layers. As illustrated in FIG. 3C, the one or more airbags 302 may include a first layer and a second layer. Moreover, the first layer may include airbag 302a, airbag 302B and airbag 302C, as similarly illustrated in FIG. 3B. Further, the second layer may include airbag 332, airbag 334, airbag 336 and airbag 338, as illustrated in FIG. 3D (e.g., second layer 330). In some aspects, airbag 332 may be positioned over the top left portion of airbag 302B, airbag 334 may be positioned over top right portion of airbag 302C, and airbag 336 may be positioned over bottom left portion of airbag 302B and airbag 338 may be positioned over bottom right portion of airbag 302B. Moreover, a pathway or fluid connection may be formed between airbag 332 and top left portion of airbag 302B, airbag 334 and top right portion of airbag 302B, airbag 336 and bottom left portion of airbag 302B, and airbag 338 and bottom right portion of airbag 302B. In such aspects, airbag 332, airbag 334, airbag 336 and airbag 336 may be inflated or deflated through nozzle 303A.

In some instances, the first layer may be between a face of a user, such as user 204, and the second layer. Alternatively, the second layer may be between the face of the user and the first layer. In some examples, combined width of airbags 302 may have a width of 22-26 cm (e.g., W1 of FIG. 3C) and a height of 7-9 cm (e.g., H1 of FIG. 3C). In some aspects, the combined width of airbags 302 may be wider to better cover the temples of a user. In such aspects, the combined width of airbags 302 may have a width of 24-30 cm and a height of 7-9 cm. In some instances, the width between nozzle 303A and 303B may be 16-20 cm (e.g., W2 of FIG. 3C). Moreover, the width between nozzle 303A or 303B to the middle of the airbags 302 may be 8-10 cm (e.g., W3 of FIG. 3C). Further, the width between the pathway or fluid connection of airbag 336 or airbag 338 and the middle of the airbags 302 may be 3-5 cm (e.g., W4 of FIG. 3C).

Referring back to FIG. 3A, housing element 300 may include a cover, a padding element and a heating device. In such cases, housing element 300 may include a front side and a back side. In some instances, the front side of housing element 300 may couple or attach to housing element 304 and housing element 325. In such instances, housing element 300 may couple to housing element 325 by one or more snap fit elements and/or one or more coupling elements. In some aspects, the front side of housing element 300 may couple to housing element 325. In such aspects, housing element 304 and the airbags 302 maybe held in place between housing element 300 and housing element 325. In some instances, the back side of housing element 300 may include the cover. The cover may be formed from material such as, but not limited to, leather, pleather, or cotton. Moreover, the cover may fully or partially cover the back side of housing element 300. As described herein, the cover is the material that the face of the user comes in contact with when wearing and using eye massager 202 (e.g., getting an eye massage from eye massager 202). In some instances, housing element 300 may have a width of 24-30 cm and a height of 7-9 cm.

In some examples, padding element may be dimensioned to fit within housing element 300 (e.g., within a frame of housing element 300). Moreover, padding element may be positioned between the cover of housing element 300 and the airbags 302 of eye massager 202 (e.g., airbag 302A, airbag 302B and airbag 302C). That way the padding element may provide additional comfort to the user while pressure is being provided by the airbags 302 of eye massager 202 through the padding element without preventing the provided pressure from being felt by the user. In such examples, the padding element may have a thickness of 0.3-1.5 cm. Moreover, the padding element may have a width of 11-15 cm and a height of 7-9 cm.

As described herein, the heating device may be configured to provide or emit heat to the user. Moreover, the heating device of eye massager 202 may have the same functionalities and perform the same processes as the heating device of heating component(s) 104. For instance, and as described herein, each mode of massage or a heating operation may include heating or temperature settings or parameters. In some instances, the heating settings or parameters may indicate a particular heat level or temperature. Based on such settings or parameters, the control engine may electrically control the heating device, via the one or more processors, to cause the heating device to output heat at the heat level or temperature as indicated by such heating settings. In some instances, the heating settings or parameters may indicate a particular duration of the heating operations. Based on such settings or parameters, the control engine may electrically or electronically control the heating device, via the one or more processors, to output heat for the duration indicated by such heating settings.

FIG. 3E illustrates an example heating device, such as heating device 340. As illustrated in FIG. 3E, heating device 340 may include heating element 342. In some aspects, heating element 342 may include a material formed out of a material with high heat conductivity and/or melting point, such as, but not limited to, copper, brass, silver, aluminum, gold, nickel, tungsten and carbon fiber. In some cases, and as illustrated in FIG. 3E, heating device 340 may include container 344 formed out of a material with high heat conductivity and/or melting point. For instance, and as illustrated in FIG. 3E, container 344 may be a film, such as a polyimide film, with heating element 342 (e.g., copper or carbon fiber material). Further, heating device 340 may include connector 346 that enables the control engine of eye massager 202, via the one or more processors of eye massager 202, to electrically or electronically control heating device 340, as described herein.

Referring back to FIG. 3A, and in some examples, the heating device may be within the padding element of eye massager 202. In such cases, the heating device may have a thickness of 0.05-0.25 mm. For instance, the heating device may be a film having a thickness of 0.15 mm formed out of a high heat conductivity material and/or melting point including a material with high heat conductivity and/or melting point (e.g., a polyimide film with copper or carbon fiber within the polyimide film). That way the pressure that is being provided by the airbags 302 of eye massager 202 may not be inhibited by the heating device. In some instances, the heating device may provide heat the user while the airbags 302 are providing pressure or massaging the user.

In some examples, eye massager 202 may include a removable cooling component (e.g., a cooling gel). As described herein, the removable cooling component, may be affixed or coupled to the back side of housing element 300. For instance, housing element 300 may include a top portion and/or a bottom portion. Moreover, housing element 300 may include one or more coupling elements (e.g., a hook and loop element). The top portion and/or the bottom portion may include the one or more coupling elements. Further, the removeable cooling component may include a cover with one or more coupling elements positioned on the edge or lip of the cover. The removable cooling component may couple to or affix to the top portion and/or bottom portion of housing element 300 by coupling the coupling elements of the cover to corresponding coupling elements on the top and/or bottom portion of housing element 300. In some instances, one or more portions of the cover of removeable cooling component may cover and wrap one or more sides of housing element 300 (e.g., elastic and flexible material that may wrap around housing element 300.

In some cases, the removable cooling component may help reduce swelling and eye puffiness a user may be experiencing. In such cases, the removable cooling component may include material that can absorb a considerable amount of heat before warming (e.g., warming above 32 degrees Fahrenheit). Moreover, the removable cooling component may be stored in a cooling device, such as a refrigerator or freezer for a predetermined amount of time (e.g., between 5 and 24 hours). While in the cooling device, such as a refrigerator or freezer, the removable cooling component may cool down and when removed from the cooling device may maintain cooling temperatures (e.g., 17 degrees Fahrenheit or −8.3 degrees Celsius) for at least a predetermined amount of time (e.g., 5 minutes).

In some instances, the removable cooling component may have a thickness of 1-3 millimeters. That way, in instances where one or more airbags 302 (e.g., airbag 302A, airbag 302B and airbag 302C) of eye massager 202 are at least inflated, the pressure exerted from and by the airbags 302 may still be felt by a face of the user (e.g., user 204 of FIG. 2) while feeling the cooling effects of the removable cooling component. In some cases, the removable cooling component may have a width of 11-15 cm and a height of 7-9 cm.

FIG. 3F illustrates an exploded view of the components included in eye massager 202. Further, FIG. 3F illustrates a simplified version of eye massager 202 in FIG. 3A, where certain structural or functional details in FIG. 3A are omitted for clarity and ease of understanding. FIG. 3F illustrates the overall configuration or structural layout of eye massager 202 while omitting the internal components such as, housing element 304, one or more compartments or airbags 302 (e.g., airbag 302A, airbag 302B, and airbag 302C), one or more nozzles (e.g., nozzle 309, nozzle 310, and nozzle 311), speaker 312, controller device 313, one or more tubing or hose (e.g., hose 314), one or more motors (e.g., vibration motor 315, vibration motor 319, and air pump motor 318), one or more valves (e.g., valve 316 and valve 317), electric charging connector 320, and one or more input devices 321.

As illustrated in FIG. 3F, eye massager 202 may include display device 350. Display device 350 may have similar functions and configurations as one or more components of display system 117, such as the display device of display system 117 as described in FIG. 1. For example, display device 350 may display state information of eye massager 202. As described herein, state information may be associated with biometric information associated with a user using or operating eye massager 202 (e.g., heart rate, skin conductance or electrodermal activity, and facial temperature), a state of the user (e.g., stressed, relax, asleep, a level of stress, etc.), one or more operations (e.g., parameter(s) or setting(s) of massage operation(s), heating operation(s) and/or vibration setting(s) that eye massager 202 may perform, such as a massage duration or time remaining, a massage pressure level, a vibrational frequency level, a heating level or temperature, etc., or corresponding adjustments) or a mode of massage that eye massager 202 may be performing or implementing.

Display device 350 may be coupled to eye massager 202. In some examples, display device 350 may be coupled to housing element 325 (e.g., one or more portions of housing element 325). For instance, display device 350 may be coupled to the front surface or side of housing element 325 (e.g., side facing away from the user when the user is wearing eye massager 202). Additionally, or alternatively, display device 350 may be coupled to housing element 322. For instance, display device may be coupled to the back surface or side of housing element 322 (e.g., side facing towards the user when the user is wearing eye massager 202). In another instance, display device may be coupled to the front surface or side of housing element 322 (e.g., side facing away the user when the user is wearing eye massager 202). In some aspects, display device 350 may attach to housing element 325 or housing element 322 by one or more coupling elements, such as one or more snap fit elements (e.g., a cantilever type snap fit element (e.g., a cantilever beam and a tapered hook), an annular type snap fit element (e.g., a circular or ring-shaped protrusion component that couples to a corresponding groove or channel component) and/or a torsional type snap fit element, a screw, magnetic elements and/or adhesive elements (e.g., a glue or adhesive tape). In some examples, and as illustrated in FIGS. 3A and 3F, housing element 322 may be coupled to housing element 325 and display device 350 may be between housing element 322 and housing element 325 (e.g., the display screen of display device 350 may be facing away the user when the user is wearing eye massager 202). In such examples, housing element 322 may be transparent or opaque to enable users to see state information being displayed or presented by display device 350 through housing element 322.

In some cases, the size, shape and/or dimensions, including the width and length, of display device 350 may vary with respect to housing element 322 and/or housing element 325. The size of display device 350 may be the same as, similar to, smaller than or larger than the size of housing element 322 and/or housing element 325. In some examples, the dimensions of display device 350 may match or substantially correspond to the dimensions of housing element 322 and/or housing element 325 (e.g., within +/−0.1 cm). For example, when the centers of display device 350 and housing element 322 are aligned, the edges of display device 350 may be aligned with the edges of housing element 322. In another example, when the centers of display device 350 and housing element 325 are aligned, the edges of display device 350 may be aligned with the edges of housing element 325.

In some aspects, the dimensions of display device 350 may be smaller than housing element 322 and/or housing element 325. For instance, when display device 350 is coupled to housing element 322, the width of the space between an edge of housing element 322 and the corresponding edge of display device 350 may range from 0 mm to 20 mm. In another instance, when display device 350 is coupled to housing element 325, the width of the space between an edge of housing element 325 and the corresponding edge of display device 350 may range from 0 mm to 20 mm. In such aspects, the coupling of display device 350 to housing element 322 and/or housing element 325 may be center aligned or non-center aligned. In instances where the coupling between display device 350 and housing element 322 and/or housing element 325 is center-aligned, the width of space between the edges of housing element 322 and/or housing element 325 and display device 350 may range from 0.1 mm to 22 mm. In some cases, the space may accommodate one or more coupling elements that enable housing element 322 to couple with housing element 325. In instances where the coupling between display device 350 and housing element 322 and/or housing element 325 are non-center-aligned, when display device 350 is coupled to housing element 322 and/or housing element 325 their centers may not be aligned. For instance, display device 350 may be offset to one side of housing element 322 or housing element 325. For example, display device 350 is offset to the left side of housing element 322. In such an example, the width of the space between the left edge of display device 350 and the left edge of housing element 322 may range from 0.1 mm to 22 mm while the width of the space between the right edge of display device 350 and the right edge of housing element 322 may range from 148 mm to 172 mm. In another example, display device 350 is offset to the right side of housing element 325. In such an example, the width of the space between the right edge of display device 350 and the right edge of housing element 322 may range from 0.1 mm to 22 mm while the width of the space between the left edge of display device 350 and the left edge of housing element 322 may range from 148 mm to 172 mm.

In some cases, the dimensions of display device 350 may be larger than housing element 322 and/or housing element 325. For example, when display device 350 and housing element 322 are coupled together and the centers of display device 350 and housing element 322 are aligned, the edges of display device 350 may extend beyond the edges of housing element 322 by 0 mm to 50 mm. In another example, when display device 350 and housing element 325 are coupled together and the centers of display device 350 and housing element 325 are aligned, the edges of display device 350 may extend beyond the edges of housing element 325 by 0 mm to 50 mm.

In some instances, the shape of display device 350 may be similar or correspond to the shape of housing element 322 and/or housing element 325. For example, as illustrated in FIG. 3F, the shape of housing element 322 and/or 325 are similar to the shape of display device 350. In some cases, the shape of display device 350 may or differ from the shape of housing element 322 and/or housing element 325. For example, display device 350 may be circular or triangular while housing element 322 and/or housing element 325 are rectangular. Examples of shapes that display device 350 may be, include, but are not limited to, a rectangular shape, a circular shape, a triangular shape or any other polygonal shape (e.g., square, hexagonal, or any other geometric shape). In some examples, the size, shape and dimensions of display device 350 may be further customized to align with ergonomic considerations, aesthetic preferences, or functional requirements. For example, display device 350 may feature a rectangular, square, or curved profile to conform to the contours of eye massager 202.

In some instances, one or more components of eye massager 202 may be modular or removable. In some aspects, housing element 322 may be removable. In such aspects, housing element 322, as described herein, may attach to housing element 325 by one or more modular coupling elements, such as one or more snap fit elements (e.g., male snap tabs may be coupled to housing element 322 and may engage with female slots or recesses on housing element 325), magnetic elements, hook and loop fastener elements Such modular coupling element(s) may enable housing element 322 to interlock securely with housing element 325 while remaining removable without the need for specialized tools. Additionally, or alternatively, the modular coupling element(s) may include other mechanisms, such as magnetic couplings, screws, or press-fit arrangements.

FIG. 3G, FIG. 3H, FIG. 3I, FIG. 3J, FIG. 3K, FIG. 3L, FIG. 3M, FIG. 3N and FIG. 3O illustrate examples of modular coupling elements. FIG. 3G, FIG. 3H, FIG. 3I, FIG. 3J, FIG. 3K, FIG. 3L, FIG. 3M, FIG. 3N and FIG. 3O each illustrate a semi-exploded view of eye massager 202. FIG. 3G, FIG. 3H, and FIG. 3I illustrate a semi-exploded view of eye massager 202 that includes hook and loop fastener element(s) to couple housing element 322 and housing element 325 together. As illustrated in FIG. 3G, FIG. 3H, FIG. 3I, housing element 325 and housing element 322 may each include hook and loop fastener element 362 (e.g., hook and loop fastener element 362A and hook and loop fastener element 362B). In some instances, hook and loop fastener element 362A may be one or more hook elements while hook and loop fastener element 362B may include one or more loop elements. In some cases, hook and loop fastener element 362A may be one or more loop elements while hook and loop fastener element 362B may include one or more hook elements.

FIG. 3J, FIG. 3K, and FIG. 3L illustrate a semi-exploded view of eye massager 202 that includes one or more snap fit elements. As illustrated in FIG. 3J, FIG. 3K, and FIG. 3L, housing element 325 and housing element 322 may each include one or more snap fit elements 364 (e.g., snap fit element(s) 364A and snap fit element(s) 364B). In some instances, each snap fit element 364A may be include a snap feature (e.g., a protrusion with a hook element) while each snap fit element 364B may include a receptor dimensioned or configured to receive a corresponding snap fit element 364A. In some aspects, each snap fit element 364A may be include the receptor while each snap fit element 364B may include the snap feature. The receptor may include a depression or groove that catches the corresponding snap feature when inserted into the receptor.

As illustrated in FIG. 3M, FIG. 3N, FIG. 3O, housing element 325 and housing element 322 may each include one or more magnetic elements. For example, as illustrated in FIG. 3M, FIG. 3N, FIG. 3O, housing element 325 may include magnetic elements 366A. Further, housing element 322 may include magnetic elements 366B.

In some examples, and as illustrated in FIG. 3F, padding element 352, similar to the padding element of FIG. 3A, may be removable. In such examples, padding element 352 may attach to housing element 300 by one or more modular coupling elements. Examples of modular coupling element(s) that couple padding element 352 to housing element 300 include, but are not limited to, hook and loop fastener elements, one or more snap buttons, and one or more magnetic mounts. In some instances, padding element 352 may include a heating device, similar to heating device 340 of FIG. 3E. In such instances, padding element 352 may include one or more electrical interfaces to electrically couple the heating device to controller device 313 and/or power source component(s) of eye massager 202. Examples of electrical interface(s) include, but are not limited to push-in connectors (e.g., two or three port push-in electrical connectors) and pogo-pin connectors (e.g., spring-loaded contact pins that establish a reliable connection between the heating device and controller device 313 and/or power source component(s) of eye massager 202 when padding element 352 is attached to housing element 300).

In some aspects, strap component 301 may be removable. As illustrated in FIG. 3F, strap component 301 may be coupled to housing element 300 via removable connectors, such as removable connector 354 and removable connector 356. As illustrated in FIGS. 3P and 3R (as well as FIG. 3F), the removable connector(s) of strap component 301 may interface with, connect, couple to housing element 300 via port(s) of housing element 300. For example, as illustrated in FIGS. 3P and 3R, removable connector 354 may couple to housing element 300 via port 359 and removable connector 356 may couple to housing element 300 via port 360. Although, FIG. 3F illustrates strap component 301 being coupled to housing element 300 via two removable connectors, strap component 301 may be coupled to housing element via one removable connector. For example, strap component 301 may include removable connector 354 while the other side of strap component 301 (where removable connector 356 is illustrated) may be fixed to housing element 300 (not removable). Removable connector 354 may couple to housing element via port 359. Alternatively, strap component 301 may include removable connector 356 while the other side of strap component 301 (where removable connector 354 is illustrated) may be fixed to housing element 300 (not removable). Removable connector 356 may couple to housing element via port 360.

In some examples, removeable connector(s) of strap component 301 and port(s) of housing element 300 may include one or more coupling elements that enable secure attachment between the removable connector(s) and the port(s). In some cases, the coupling element(s) may include one or more latch mechanisms. A latch mechanism may include a button and a latch element. When the button is compressed the latch element may be in a disengaged position (e.g., retract partially or fully into the corresponding removable connector). When the button is not compressed, the latch element may be in an engaged position (e.g., extended partially or fully out of the corresponding removable connector). If the portion of the removable connector with the engaged latch element is in the corresponding port, the engaged latched element may secure the removable connector to the corresponding port by engaging with a corresponding recess or slot within the port. In some instances, the removable connector(s) may include a single latch mechanism. FIG. 3F, FIG. 3G, FIG. 3H, FIG. 3I, FIG. 3J, FIG. 3K, FIG. 3L, FIG. 3M, FIG. 3N and FIG. 3O all illustrate removeable connector(s) with a single latch mechanism.

For example, as illustrated in FIG. 3F, FIG. 3G, FIG. 3H, FIG. 3I, FIG. 3J, FIG. 3K, FIG. 3L, FIG. 3M, FIG. 3N, FIGS. 3O and 3R, removable connector 354 may include button 357 and latch element 358. As described herein, port 359 may include a slot or recess dimensioned to fit latch element 358. When button 357 is compressed, latch element 358 may be in a disengaged position (e.g., retract partially or fully into removable). When one or more portions of removable connector 354 is inserted into port 359 and button 357 is not compressed, latch element may be in an engaged position and engage with the slot or recess of port 359.

In some aspects, the removable connector(s) may include multiple latch mechanisms. FIG. 3P illustrates a semi-exploded view of eye massager 202 with removable connector(s) with multiple latch mechanisms. As illustrated in FIG. 3P, removable connector 354 may include button 368A, latch element 368B, button 368C and latch element 368D, and removable connector 356 may include button 369A, latch element 369B, button 369C and latch element 369D. Port 359 may include slots or recesses dimensioned to fit latch element 368B and latch element 368D. Port 360 may include slots or recesses dimensioned to fit latch element 369B and latch element 369D.

For example, when button 368A and button 368C are compressed, latch element 368B and latch element 368D may each be in a disengaged position (e.g., retract partially or fully into removable). When one or more portions of removable connector 354 is inserted into port 359 and button 368A and button 368C are not compressed, latch element 368B and latch element 368D may each be in an engaged position and engage with the slots or recesses of port 359.

In some instances, the coupling element(s) may include guiding elements. The guiding elements may prevent misalignment that could damage internal components when removable connector(s) are being inserted into corresponding port(s). Example of guiding elements include one or more channel(s) and/or one or more rail(s). For example, removable connector 354 and removable connector 356 may each include a channel or recess along their bottom surface (not illustrated in FIG. 3F). Port 359 and port 360 may each include a rail (not illustrated in FIG. 3F). The channel of removable connector 354 may be configured or designed to slide over the rail of port 359. The channel of removable connector 356 may be configured or designed to slide over the rail of port 360.

In some cases, the coupling element(s) may include an ejection assist system. In some instances, the ejection assist system may be integrated into the port(s) of housing element 304. In such instances, the ejection assist system may include a spring-loaded ejection mechanism. For instance, when a removable connector (e.g., removable connector 354) with a latch mechanism is inserted into a port (e.g., 359) with the spring-loaded ejection mechanism, the spring-loaded ejection mechanism compresses. When the latch mechanism is engaged (e.g., extended) with a slot or recess of the port, the removable connector is secured in place with the spring-loaded ejection mechanism compressed pushing against the removable connector. When the latch mechanism is disengaged (e.g., retracted) the spring-loaded ejection mechanism expands, pushing the removable connector out of the port for easy removal.

FIG. 3Q and FIG. 3R illustrates a rear view of an example electronic eye massager. As illustrated in FIG. 3Q and FIG. 3R, one or more sensing components, such as sensing component 370. The sensing components may be may be positioned on the rear of eye massager 202 to interface with one or more portions of a head of a user operating or using eye massager 202. As similarly described herein with the one or more sensing component(s) 116 of FIG. 1, the sensing components may be configured to interface with one or more portions of a head and/or face of a user using eye massager 202. Sensing components may generate sensor data of the user, as similarly described herein with the one or more sensing component(s) 116 of FIG. 1. In some examples, controller device 313, similar to control engine 108, may determine biometric information of the user based on the sensor data. In some instances, controller device 313, similar to control engine 108, may implement processes, as similarly described with control engine 108, to initiate the massage operation(s) or the mode of massage based on the sensor data. Additionally, or alternatively, controller device 313, similar to control engine 108, may implement processes, as similarly described with control engine 108, to adjust the parameter(s) or setting(s) of the massage operation(s) or the mode of massage currently being performed by eye massager system 100 based on the sensor data. Further, controller device 313, as similarly described with control engine 108, may implement processes to provide state information based on sensor data of the user to display device 350.

In some examples, the sensing component(s), such as sensing component 370, may be coupled or attached to padding element 352. The sensing component(s) may be embedded within the padding material of padding element 352, or affixed to the inner surface of padding element 352 to maintain contact with the user's skin during use of eye massager 202. For instance, adhesive, stitching, or integrally molded features may secure the sensing component(s)to the padding. Alternatively, padding element 352 may include one or more cutouts, apertures, or holes specifically designed to the sensing component(s). The cutout(s) are positioned to align the sensing component(s) with a corresponding contact area on the user's head or face. The sensing component(s) may protrude partially or fully through the cutout to ensure proper engagement with the user's skin. Padding element 352 may be constructed from a flexible material that conforms around the sensor, providing comfort while ensuring consistent positioning. Although FIG. 3Q and FIG. 3R illustrate an example placement of the sensing component, it should be understood that the sensing component and/or cutout(s) for the sensing component may be positioned at any location on padding element 352 provided that the sensing component can interface with the face or head of the user.

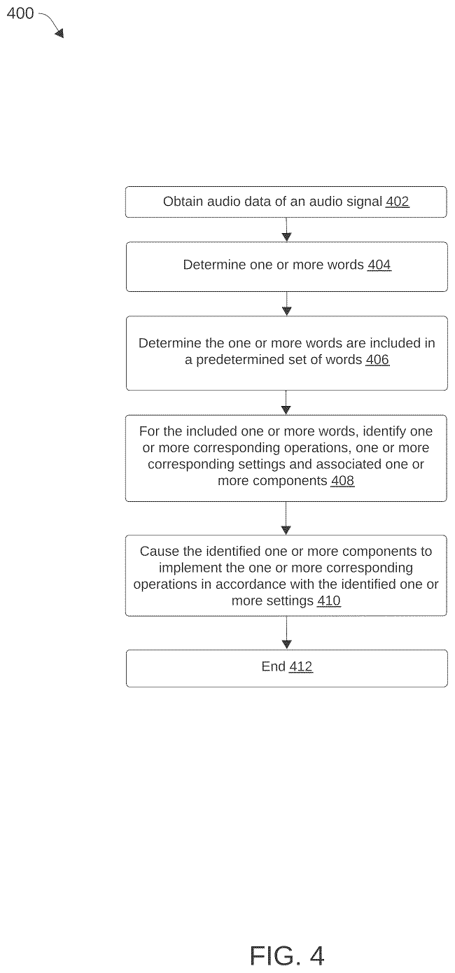

FIG. 4 is a flow chart for a method 400 for controlling the example electronic eye massager with spoken commands, according to some examples of the present disclosure. Method 400 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously, or in a different order than shown in any figures of the disclosure, as will be understood by a person of ordinary skill in the art.

Method 400 shall be described with reference to FIG. 1-3. However, method 400 is not limited to that example. At step 402, voice recognition engine 110 may obtain audio data of an audio signal obtained by microphone 112. As described herein, microphone 112 may detect and obtain audio signals from a user and generate the audio data based on the obtained audio signals. Moreover, the audio data may identify and/or the audio signal may indicate one or more words, phrases or terms spoken by the user. Further, microphone 112 may generate the audio data based on the detected or obtained audio signal. In some instances, microphone 112 may provide the audio data to voice recognition engine 110.

At step 404, voice recognition engine 110 may determine the one or more words, phrases or terms, spoken by the user based on the audio data. Moreover, voice recognition engine 110 may generate term data including the determined words, phrases, or terms spoken by the user. In some instances, voice recognition engine 110 may generate term data including the determined words, phrases or terms. Moreover, the voice recognition engine may transmit or provide the term data to control engine 108.

In some aspects, voice recognition engine 110 may apply one or more AI/ML models, such as a speech recognition-based AI/ML model, to the audio data. Based on the application of the one or more AI/ML models to the audio data, voice recognition engine 110 may perform any of the described examples processes to determine one or more words, phrase or terms spoken by the user. In such aspects, voice recognition engine 110 may generate term data including the determined one or more words, phrases or terms spoken by the user.

At step 406, control engine 108 may determine the one or more words, phrases or terms spoken by the user are included in a predetermined set of words, phrases and/or terms. In some cases, control engine 108 may determine whether the one or more words, phrases or terms spoken by the user match a known or predetermined set of words, phrases and/or terms based on the term data and command data. In such cases, control engine 108 may access the command data stored in database 111 to determine whether the one or more words, phrases or terms spoken by the user match a known or predetermined set of words, phrases and/or terms included in the term data.

As described herein, the command data may identify one or more predetermined words, phrases and/or terms, one or more corresponding types of operations eye massager system 100 may perform (e.g., a massage operation, a vibration operation and/or heating operation) for each of the one or more predetermined words, phrases and/or terms, one or more parameters or settings of each corresponding types of the operations, and, for each of the parameters or settings, corresponding components (e.g., components of massage component(s) 102, heating component(s) 104 and/or vibration component(s) 106). Examples of parameters or settings that may be identified in the command data may include, but are not limited to, an inflation and/or deflation pattern, level of pressure (e.g., pounds per square inch or “psi”), duration of massage operation, a vibrational pattern, level of frequency, duration of vibrational operation, heating level, and duration of heating operation.

At step 408, control engine 108 may, for the included one or more words, identify one or more corresponding operations, corresponding one or more settings, and associated one or more components. In some instances, control engine 108 may identify the one or more corresponding operations, corresponding one or more settings, and one or more associated components based on the command data. As described herein, the included or matched one or more words may be associated with a mode of massage, changing or switching the mode of massage eye massager system 100 may be performing or implementing to another mode of massage, or one or more adjustments to one or more settings of a mode of massage, a massage operation, a heating operation and/or a vibrational operation (e.g., a massage setting, a heating setting and/or a vibrational setting).

At step 410, control engine may cause the identified one or more components to implement the corresponding operation in accordance with the identified one or more settings. In some examples, control engine 108 may electrically or electronically communicate with each of the identified components, via the one or more processors of eye massager 100, to cause each of the identified components to perform or implement the identified operation in accordance with the corresponding identified parameters and settings (e.g., increase or decrease electrical power to the components in accordance with the identified parameters and settings).

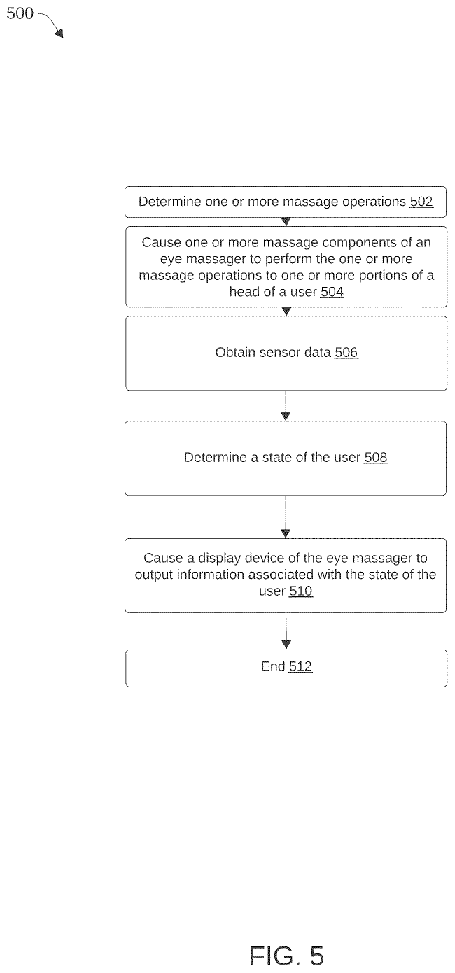

FIG. 5 is a flow chart for a method 500 for providing a state of a user, via a display device of an eye massager, while the eye massager performs one or more massage operations on one or more portions of a head or face of a user, according to some examples of the present disclosure. Method 500 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously, or in a different order than shown in any figures of the disclosure, as will be understood by a person of ordinary skill in the art.

Method 500 shall be described with reference to FIG. 1-3H. However, method 500 is not limited to that example. At step 502, control engine 108 may determine one or more massage operations. In some examples, control engine 108 may determine the one or more massage operations based on audio data. In some instances, control engine 108 may determine the one or more massage operations based on sensor data generated by one or more sensing components of eye massager system 100.

At step 504, control engine 108 may cause one or more massage components of an eye massager to perform the one or more massage operations to one or more portions of a head of a user. As described herein, control engine 108 may electrically or electronically communicate with massage component(s) 102 to cause massage component(s) 102 to perform or implement the determined one or more massage operations.

At step 506, control engine 108 may obtain sensor data. At step 508, control engine 108 may determine a state of the user based on the sensor data. In some examples, control engine 108 may obtain the sensor data from one or more sensing components of eye massager system 100. In some aspects, control engine 108 may determine biometric information associated with a user using eye massage system 100. Examples of biometric information control engine 108 may determine may include, but is not limited to, heart rate, blood oxygen levels, blood pressure, respiration, other cardiovascular variability assessments, skin conductance or electrodermal activity, and facial temperature. In some instances, control engine 108 may determine the state of the user based on the biometric information of the user. As described herein, the state of the user may indicate a stress level of the user while the one or more massage components perform the one or more massage operations to the one or more portions of the head of the user.

At step 510, control engine 108 may cause a display device of the eye massager to output information associated with the state of the user. In some examples, control engine 108 may provide information of the state of the user to the display device, such as display device 350. Display device 350 may display the information of the state of the user. Examples of a display device that display system 117 may include, but are not limited to, a light emitting diode (LED) dot matrix or matrix panel, a liquid-crystal display (LCD) screen, an LED screen and an organic LED (OLED) screen.

As described herein, the display device and/or display system 117 may display or present the state information, via one or more formats. Examples of formats the display device and/or display system 117 may present the state information, includes, but is not limited to, color coded visual indicators (e.g., green light indicates a relaxed or calm state, red light indicates elevated stress, blue light indicates a state of deep relaxation or sleep, etc.), graphics/images, such as emoticons or symbols (e.g., a smiling face may indicate relaxation while a frowning face or exclamation mark may indicate stress or tension), textual output (e.g., alphanumeric or textual descriptions corresponding to the state of the user, such as “relaxed” to indicate a low-stress state or “stressed” to indicate a user is in a stressed state), and animations/videos (e.g., videos or animations correlating to the state of the user, such as a wave-like animation to indicate the user is relaxed, a flashing red animation to indicate the user is stressed or has elevated stress levels, etc.).

### Example Neural Network Architectures

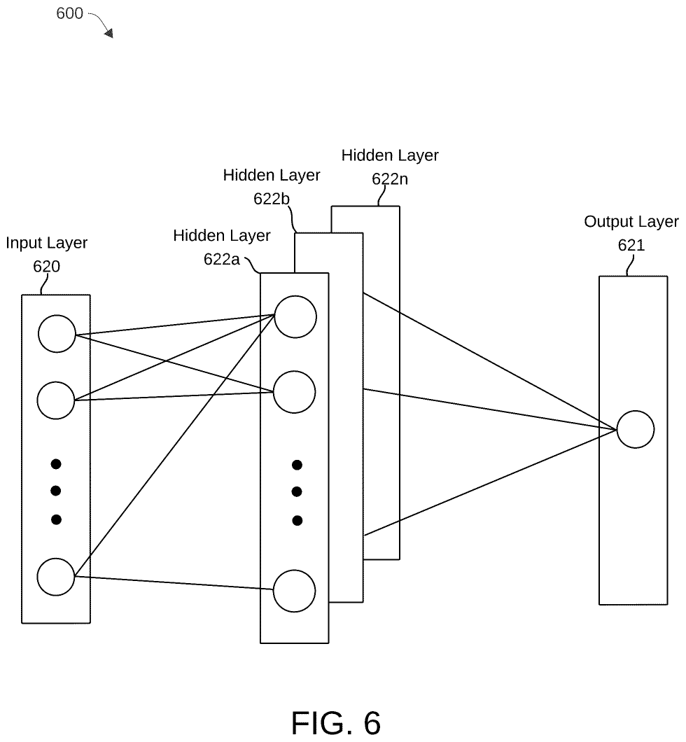

FIG. 6 is a diagram illustrating an example of a neural network architecture 600 that can be used to implement some or all of the neural networks described herein. The neural network architecture 600 can include an input layer 620 that can be configured to receive and process data to generate one or more outputs. The neural network architecture 600 also includes hidden layers 622a, 622b, through 622n. The hidden layers 622a, 622b, through 622n include “n” number of hidden layers, where “n” is an integer greater than or equal to one. The number of hidden layers can be made to include as many layers as needed for the given application. The neural network architecture 600 further includes an output layer 621 that provides an output resulting from the processing performed by the hidden layers 622a, 622b, through 622n.

The neural network architecture 600 is a multi-layer neural network of interconnected nodes. Each node can represent a piece of information. Information associated with the nodes is shared among the different layers and each layer retains information as information is processed. In some cases, the neural network architecture 600 can include a feed-forward network, in which case there are no feedback connections where outputs of the network are fed back into itself. In some cases, the neural network architecture 600 can include a recurrent neural network, which can have loops that allow information to be carried across nodes while reading in input.

Information can be exchanged between nodes through node-to-node interconnections between the various layers. Nodes of the input layer 620 can activate a set of nodes in the first hidden layer 622a. For example, as shown, each of the input nodes of the input layer 620 is connected to each of the nodes of the first hidden layer 622a. The nodes of the first hidden layer 622a can transform the information of each input node by applying activation functions to the input node information. The information derived from the transformation can then be passed to and can activate the nodes of the next hidden layer 622b, which can perform their own designated functions. Example functions include convolutional, up-sampling, data transformation, and/or any other suitable functions. The output of the hidden layer 622b can then activate nodes of the next hidden layer, and so on. The output of the last hidden layer 622n can activate one or more nodes of the output layer 621, at which an output is provided. In some cases, while nodes in the neural network architecture 600 are shown as having multiple output lines, a node can have a single output and all lines shown as being output from a node represent the same output value.

In some cases, each node or interconnection between nodes can have a weight that is a set of parameters derived from the training of the neural network architecture 600. Once the neural network architecture 600 is trained, it can be referred to as a trained neural network, which can be used to generate one or more outputs. For example, an interconnection between nodes can represent a piece of information learned about the interconnected nodes. The interconnection can have a tunable numeric weight that can be tuned (e.g., based on a training dataset), allowing the neural network architecture 600 to be adaptive to inputs and able to learn as more and more data is processed.

The neural network architecture 600 is pre-trained to process the features from the data in the input layer 620 using the different hidden layers 622a, 622b, through 622n in order to provide the output through the output layer 621.

In some cases, the neural network architecture 600 can adjust the weights of the nodes using a training process called backpropagation. A backpropagation process can include a forward pass, a loss function, a backward pass, and a weight update. The forward pass, loss function, backward pass, and parameter/weight update is performed for one training iteration. The process can be repeated for a certain number of iterations for each set of training data until the neural network architecture 600 is trained well enough so that the weights of the layers are accurately tuned.

To perform training, a loss function can be used to analyze an error in the output. Any suitable loss function definition can be used, such as a Cross-Entropy loss. Another example of a loss function includes the mean squared error (MSE), defined as E_total=Σ(½ (target−output)\hat{ }2). The loss can be set to be equal to the value of E_total.

The loss (or error) will be high for the initial training data since the actual values will be much different than the predicted output. The goal of training is to minimize the amount of loss so that the predicted output is the same as the training output. The neural network architecture 600 can perform a backward pass by determining which inputs (weights) most contributed to the loss of the network and can adjust the weights so that the loss decreases and is eventually minimized.

The neural network architecture 600 can include any suitable deep network. One example includes a Convolutional Neural Network (CNN), which includes an input layer and an output layer, with multiple hidden layers between the input and out layers. The hidden layers of a CNN include a series of convolutional, nonlinear, pooling (for downsampling), and fully connected layers. The neural network architecture 600 can include any other deep network other than a CNN, such as an autoencoder, Deep Belief Nets (DBNs), Recurrent Neural Networks (RNNs), among others.

As understood by those of skill in the art, machine-learning based techniques can vary depending on the desired implementation. For example, machine-learning schemes can utilize one or more of the following, alone or in combination: hidden Markov models; RNNs; CNNs; deep learning; Bayesian symbolic methods; Generative Adversarial Networks (GANs); support vector machines; image registration methods; and applicable rule-based systems. Where regression algorithms are used, they may include but are not limited to: a Stochastic Gradient Descent Regressor, a Passive Aggressive Regressor, etc.

Machine learning classification models can also be based on clustering algorithms (e.g., a Mini-batch K-means clustering algorithm), a recommendation algorithm (e.g., a Minwise Hashing algorithm, or Euclidean Locality-Sensitive Hashing (LSH) algorithm), and/or an anomaly detection algorithm, such as a local outlier factor. Additionally, machine-learning models can employ a dimensionality reduction approach, such as, one or more of: a Mini-batch Dictionary Learning algorithm, an incremental Principal Component Analysis (PCA) algorithm, a Latent Dirichlet Allocation algorithm, and/or a Mini-batch K-means algorithm, etc.

### Example Computer System

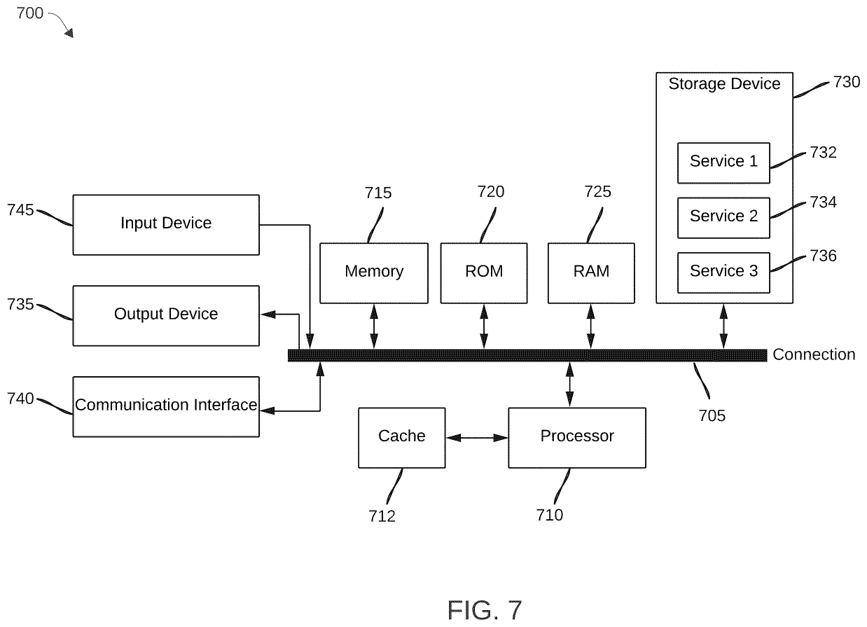

Various aspects and examples may be implemented, for example, using one or more well-known computer systems, such as computer system 700 shown in FIG. 7. For example, the eye massager system 100 and/or eye massager 202 may be implemented using combinations or sub-combinations of computer system 700. Also, or alternatively, one or more computer systems 700 may be used, for example, to implement any of the aspects and examples discussed herein, as well as combinations and sub-combinations thereof.

Computer system 700 may include one or more processors (also called central processing units, or CPUs), such as a processor 704. Processor 704 may be connected to a communication infrastructure or bus 706.

Computer system 700 may also include user input/output device(s) 703, such as monitors, keyboards, pointing devices, etc., which may communicate with communication infrastructure 706 through user input/output interface(s) 702.

One or more of processors 704 may be a graphics processing unit (GPU). In some examples, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 700 may also include a main or primary memory 708, such as random access memory (RAM). Main memory 708 may include one or more levels of cache. Main memory 708 may have stored therein control logic (e.g., computer software) and/or data.

Computer system 700 may also include one or more secondary storage devices or memory 710. Secondary memory 710 may include, for example, a hard disk drive 712 and/or a removable storage device or drive 714. Removable storage drive 714 may be a floppy disk drive, a magnetic tape drive, a compact disk drive, an optical storage device, tape backup device, and/or any other storage device/drive.

Removable storage drive 714 may interact with a removable storage unit 718. Removable storage unit 718 may include a computer usable or readable storage device having stored thereon computer software (control logic) and/or data. Removable storage unit 718 may be a floppy disk, magnetic tape, compact disk, DVD, optical storage disk, and/any other computer data storage device. Removable storage drive 714 may read from and/or write to removable storage unit 718.

Secondary memory 710 may include other means, devices, components, instrumentalities or other approaches for allowing computer programs and/or other instructions and/or data to be accessed by computer system 700. Such means, devices, components, instrumentalities or other approaches may include, for example, a removable storage unit 722 and an interface 720. Examples of the removable storage unit 722 and the interface 720 may include a program cartridge and cartridge interface (such as that found in video game devices), a removable memory chip (such as an EPROM or PROM) and associated socket, a memory stick and USB or other port, a memory card and associated memory card slot, and/or any other removable storage unit and associated interface.

Computer system 700 may include a communication or network interface 724. Communication interface 724 may enable computer system 700 to communicate and interact with any combination of external devices, external networks, external entities, etc. (individually and collectively referenced by reference number 728). For example, communication interface 724 may allow computer system 700 to communicate with external or remote devices 728 over communications path 726, which may be wired and/or wireless (or a combination thereof), and which may include any combination of LANs, WANs, the Internet, etc. Control logic and/or data may be transmitted to and from computer system 700 via communication path 726.

Computer system 700 may also be any of a personal digital assistant (PDA), desktop workstation, laptop or notebook computer, netbook, tablet, smart phone, smart watch or other wearable, appliance, part of the Internet-of-Things, and/or embedded system, to name a few non-limiting examples, or any combination thereof.

Computer system 700 may be a client or server, accessing or hosting any applications and/or data through any delivery paradigm, including but not limited to remote or distributed cloud computing solutions; local or on-premises software (“on-premise” cloud-based solutions); “as a service” models (e.g., content as a service (CaaS), digital content as a service (DcaaS), software as a service (SaaS), managed software as a service (MsaaS), platform as a service (PaaS), desktop as a service (DaaS), framework as a service (FaaS), backend as a service (BaaS), mobile backend as a service (MbaaS), infrastructure as a service (IaaS), etc.); and/or a hybrid model including any combination of the foregoing examples or other services or delivery paradigms.

Any applicable data structures, file formats, and schemas in computer system 700 may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with known or open standards.

In some examples, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 700, main memory 708, secondary memory 710, and removable storage units 718 and 722, as well as tangible articles of manufacture embodying any combination of the foregoing. Such control logic, when executed by one or more data processing devices (such as computer system 700 or processor(s) 704), may cause such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown in FIG. 7. In particular, embodiments can operate with software, hardware, and/or operating system implementations other than those described herein.

### Conclusion

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein.

References herein to “one embodiment,” “an embodiment,” “an example embodiment,” or similar phrases, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein. Additionally, some embodiments can be described using the expression “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but yet still co-operate or interact with each other.

The breadth and scope of this disclosure should not be limited by any of the above-described exemplary embodiments, but should be defined only in accordance with the following claims and their equivalents.

Claim language or other language in the disclosure reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

Illustrative examples of the disclosure include:

Aspect 1. An eye massager comprising: a display device; a first housing element; a second housing element, the display device being coupled to the second housing element and the first housing element being coupled to the second housing element with the display device being between the first housing element and the second housing element; one or more massage components; a memory storing instructions; and at least one processor coupled to the memory and configured to execute the instructions to: determine one or more massage operations for a user; cause the one or more massage components to perform the one or more massage operations to one or more portions of a head of a user; while the one or more massage components perform the one or more massage operations to the one or more portions of the head of the user, determine a state of the user; and cause the display device to output information associated with the state of the user.

Aspect 2. The eye massager of Aspect 1, further comprising one or more sensors, wherein the at least one processor is further configured to execute the instructions to: obtain sensor data generated by the one or more sensors; determine biometric information of the user based on the sensor data; and based on the biometric information, determine a state of the user at a first instance, wherein determining the one or more operations for the user is based on the state of the user at a first instance.

Aspect 3. The eye massager of any of Aspects 1 to 2, further comprising one or more sensors, wherein to determine the state of the user, the at least one processor executes the instructions to: obtain sensor data generated by the one or more sensors; determine biometric information of the user based on the sensor data; and based on the biometric information, determine the state of the user while the one or more massage components perform the one or more massage operations to the one or more portions of the head of the user.

Aspect 4. The eye massager of Aspect 3, wherein the at least one processor is further configured to execute the instructions to: determine one or more adjustments to the one or more massage operations based on the biometric information; and cause the one or more massage components to perform the one or more massage operations in accordance with the determined one or more adjustments.

Aspect 5. The eye massager of Aspect 4, wherein the at least one processor is further configured to execute the instructions to: cause the display device to output information associated with the determined one or more adjustments.

Aspect 6. The eye massager of any of Aspects 1 to 5, further comprising: one or more sensors; and a third housing element, the third housing element being coupled to the second housing element and including the one or more sensors, the one or more sensors interface with the one or more portions of the head of the user when the user is using the eye massager.

Aspect 7. The eye massager of any of Aspects 1 to 6, wherein the state of the user indicates a stress level of the user.

Aspect 8. The eye massager of any of Aspects 1 to 7, further comprising: one or more heating components, wherein the at least one processor is further configured to execute the instructions to cause the one or more heating components to perform one or more heating operations to the one or more portions of the head of the user.

Aspect 9. The eye massager of any of Aspects 1 to 8, further comprising: one or more vibrational components, wherein the at least one processor is further configured to execute the instructions to cause the one or more vibrational components to perform one or more vibrational operations to the one or more portions of the head of the user.

Aspect 10. The eye massager of any of Aspects 1 to 9, further comprising a microphone, wherein the at least one processor is further configured to execute the instructions to: obtain audio data of one or more words spoken by the user, wherein determining the one or more operations is based on the audio data.

Aspect 11. The eye massager of any of Aspects 1 to 10, wherein a width of space between an edge of the display device and an edge of second housing device is within a range of 0 mm to 20 mm.

Aspect 12. The eye massager of any of Aspects 1 to 11, wherein the first housing element is transparent.

Aspect 13. The eye massager of any of Aspects 1 to 12, wherein the first housing element is opaque.

Aspect 14. The eye massager of any of Aspects 1 to 13, wherein the first housing element is removable.

Aspect 15. An eye massager comprising: a display device; body, the display device being coupled to the body; one or more massage components; a memory storing instructions; and at least one processor coupled to the memory and configured to execute the instructions to: determine one or more massage operations for a user; cause the one or more massage components to perform one or more massage operations to one or more portions of a head of a user; while the one or more massage components perform the one or more massage operations to the one or more portions of the head of the user, determine a state of the user, the state of the user indicating a stress level of the user; and cause the display device to output information associated with the state of the user.

Aspect 16. The eye massager of Aspect 15, wherein the body includes one or more sensors, wherein the at least one processor executes the instructions to: obtain sensor data generated by the one or more sensors; determine biometric information of the user based on the sensor data; and based on the biometric information, determine a state of the user at a first instance, wherein determining the one or more operations for the user is based on the state of the user at a first instance.

Aspect 17. The eye massager of any of Aspects 15 to 16, wherein the body includes one or more sensors, wherein to determine the state of the user, the at least one processor executes the instructions to: obtain sensor data generated by the one or more sensors; determine biometric information of the user based on the sensor data; and determine the state of the user based on the biometric information.

Aspect 18. The eye massager of Aspect 17, wherein the at least one processor is further configured to execute the instructions to: determine one or more adjustments to the one or more massage operations based on the biometric information; cause the one or more massage components to perform the one or more massage operations in accordance with the determined one or more adjustments; and cause the display device to output information associated with the determined one or more adjustments.

Aspect 19. The eye massager of any of Aspects 15 to 18, wherein the body includes a first housing element and a second housing element, wherein the display device is coupled to the second housing element, and wherein the first housing element is coupled to the second housing element with the display device being between the first housing element and the second housing element.

Aspect 20. A computer-implemented method comprising: determining one or more massage operations; causing one or more massage components of an eye massager to perform the one or more massage operations to one or more portions of a head of a user; obtaining sensor data from one or more sensors of the eye massager; while the one or more massage components perform the one or more massage operations to the one or more portions of the head of the user, determining a state of the user based on the sensor data, the state of the user indicating a stress level of the user; and causing a display device of the eye massager to output information associated with the state of the user.

What is claimed is:

1. An eye massager comprising:
a display device;
a first housing element;
a second housing element, the display device being coupled to the second housing element and the first housing element being coupled to the second housing element with the display device being between the first housing element and the second housing element;
one or more massage components;
a memory storing instructions; and
at least one processor coupled to the memory and configured to execute the instructions to:
determine one or more massage operations for a user;
cause the one or more massage components to perform the one or more massage operations to one or more portions of a head of a user;
while the one or more massage components perform the one or more massage operations to the one or more portions of the head of the user, determine a state of the user; and
cause the display device to output information associated with the state of the user.

2. The eye massager of claim 1, further comprising one or more sensors, wherein the at least one processor is further configured to execute the instructions to:
obtain sensor data generated by the one or more sensors;
determine biometric information of the user based on the sensor data; and
based on the biometric information, determine a state of the user at a first instance, wherein determining the one or more operations for the user is based on the state of the user at a first instance.

3. The eye massager of claim 1, further comprising one or more sensors, wherein to determine the state of the user, the at least one processor executes the instructions to:
obtain sensor data generated by the one or more sensors;
determine biometric information of the user based on the sensor data; and
based on the biometric information, determine the state of the user while the one or more massage components perform the one or more massage operations to the one or more portions of the head of the user.

4. The eye massager of claim 3, wherein the at least one processor is further configured to execute the instructions to:
determine one or more adjustments to the one or more massage operations based on the biometric information; and
cause the one or more massage components to perform the one or more massage operations in accordance with the determined one or more adjustments.

5. The eye massager of claim 4, wherein the at least one processor is further configured to execute the instructions to:
cause the display device to output information associated with the determined one or more adjustments.

6. The eye massager of claim 1, further comprising:
one or more sensors; and
a third housing element, the third housing element being coupled to the second housing element and including the one or more sensors, the one or more sensors interface with the one or more portions of the head of the user when the user is using the eye massager.

7. The eye massager of claim 1, wherein the state of the user indicates a stress level of the user.

8. The eye massager of claim 1, further comprising:
one or more heating components, wherein the at least one processor is further configured to execute the instructions to cause the one or more heating components to perform one or more heating operations to the one or more portions of the head of the user.

9. The eye massager of claim 1, further comprising:
one or more vibrational components, wherein the at least one processor is further configured to execute the instructions to cause the one or more vibrational components to perform one or more vibrational operations to the one or more portions of the head of the user.

10. The eye massager of claim 1, further comprising a microphone, wherein the at least one processor is further configured to execute the instructions to:
obtain audio data of one or more words spoken by the user, wherein determining the one or more operations is based on the audio data.

11. The eye massager of claim 1, wherein a width of space between an edge of the display device and an edge of second housing device is within a range of 0 mm to 20 mm.

12. The eye massager of claim 1, wherein the first housing element is transparent.

13. The eye massager of claim 1, wherein the first housing element is opaque.

14. The eye massager of claim 1, wherein the first housing element is removable.

15. An eye massager comprising:
a display device;
body, the display device being coupled to the body;
one or more massage components;
a memory storing instructions; and
at least one processor coupled to the memory and configured to execute the instructions to:
determine one or more massage operations for a user;
cause the one or more massage components to perform one or more massage operations to one or more portions of a head of a user;
while the one or more massage components perform the one or more massage operations to the one or more portions of the head of the user, determine a state of the user, the state of the user indicating a stress level of the user; and
cause the display device to output information associated with the state of the user.

16. The eye massager of claim 15, wherein the body includes one or more sensors, wherein the at least one processor executes the instructions to:
obtain sensor data generated by the one or more sensors;
determine biometric information of the user based on the sensor data; and
based on the biometric information, determine a state of the user at a first instance, wherein determining the one or more operations for the user is based on the state of the user at a first instance.

17. The eye massager of claim 15, wherein the body includes one or more sensors, wherein to determine the state of the user, the at least one processor executes the instructions to:
obtain sensor data generated by the one or more sensors;
determine biometric information of the user based on the sensor data; and
determine the state of the user based on the biometric information.

18. The eye massager of claim 17, wherein the at least one processor is further configured to execute the instructions to:
determine one or more adjustments to the one or more massage operations based on the biometric information;
cause the one or more massage components to perform the one or more massage operations in accordance with the determined one or more adjustments; and
cause the display device to output information associated with the determined one or more adjustments.

19. The eye massager of claim 15, wherein the body includes a first housing element and a second housing element, wherein the display device is coupled to the second housing element, and wherein the first housing element is coupled to the second housing element with the display device being between the first housing element and the second housing element.

20. A computer-implemented method comprising:
determining one or more massage operations;
causing one or more massage components of an eye massager to perform the one or more massage operations to one or more portions of a head of a user;
obtaining sensor data from one or more sensors of the eye massager;
while the one or more massage components perform the one or more massage operations to the one or more portions of the head of the user, determining a state of the user based on the sensor data, the state of the user indicating a stress level of the user; and
causing a display device of the eye massager to output information associated with the state of the user.
